(19) United States
(12) Patent Application Publication
(10) Pub. No.: US 2025/0346642 A1
(43) Pub. Date: Nov. 13, 2025

(54) NEW SPLICE VARIANT ISOFORM OF VEGF

(71) Applicants: CENTRE NATIONAL DE LA RECHERCHE SCIENTIFIQUE (CNRS), PARIS (FR); INSERM (Institut National de la Sante et de la Recherche Medicale), PARIS (FR); UNIVERSITE COTE D'AZUR, NICE (FR)

(72) Inventors: Gilles PAGES, MONACO (MC); Jerome DURIVAULT, SAINT LAURENT DU VAR (MC); Christopher MONTEMAGNO, CHARNECLES (FR)

(21) Appl. No.: 18/250,569

(22) PCT Filed: Oct. 28, 2021
(86) PCT No.: PCT/EP2021/080033

§ 371 Date: Apr. 26, 2023

(30) Foreign Application Priority Data
Oct. 28, 2020 (EP) 20204454.1

(51) Int. Cl. C07K 14/475; A61K 38/00; A61K 45/00; C07K 16/22; C12Q 1/6886

(52) U.S. Cl. CPC C07K 14/475; A61K 45/00; C07K 16/22; C12Q 1/6886; A61K 38/00; C12Q 2600/106; C12Q 2600/156; C12Q 2600/158

(57) ABSTRACT

The present invention relates notably to a new isolated splice variant isoform of VEGF having pro-angiogenic and pro-lymphangiogenic activity, said isolated isoform comprising an amino acid sequence having at least 80%, preferably at least 95%, identity to the amino acid sequence of SEQ ID NO:1, preferably is VEGF_{222/NF} and consists of SEQ ID NO:1.

### FIELD OF THE INVENTION

The present invention relates to a new isolated splice variant isoform of VEGF having pro-angiogenic and pro-lymphangiogenic activity and for its use in the treatment of pathologies associated with insufficient angiogenesis. The invention also relates to its use as a prognostic marker and as a predictive marker of the efficacy of anti-tumoral treatment. In further aspects, the invention also relates to its related cDNA and RNA nucleotide molecule sequences, and its inhibitors for use in the prevention and the treatment of an angiogenesis-dependent disease condition.

### CROSS-REFERENCE TO RELATED APPLICATIONS

This application is a U.S. National Stage Entry of PCT/EP2021/080033, filed Oct. 28, 2021, and claims priority to European Patent Application No. 20204454.1, filed Oct. 28, 2020, incorporated by reference in its entirety.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Jun. 10, 2023, is labeled REVISED_SEQUENCE_LISTING.TXT and is 29 kilobytes in size.

### BACKGROUND

Tumors require sustained nutrients and oxygen supply and the ability to evacuate carbon dioxide and wastes. These needs are fulfilled by the tumor-associated neo-vasculature along the process of angiogenesis (Hanahan and Folkman, 1996, Patterns and emerging mechanisms of the angiogenic switch during tumorigenesis. Cell 86, 353-364).

Angiogenesis is transiently turned on in physiological processes such as female reproductive cycle or wound (Bikfalvi, 2017, History and conceptual developments in vascular biology and angiogenesis research: a personal view. Angiogenesis 20, 463-478). In contrast during tumor progression, angiogenesis is sustained to create a vascular network at the origin of tumor cells dissemination (Nishida et al., 2006, Angiogenesis in cancer. Vasc Health Risk Manag 2, 213-219). Angiogenesis is an equilibrated phenomenon involving pro- and anti-angiogenic factors. In cancer, this balance is shifted toward pro-angiogenic factors sustaining aberrant neovascularization.

In 1989, the discovery of the Vascular Endothelial Growth Factor (VEGF), one of the most important pro-angiogenic factors was a breakthrough in understanding the mechanisms of angiogenesis (Guyot and Pages, 2015, VEGF Splicing and the Role of VEGF Splice Variants: From Physiological-Pathological Conditions to Specific Pre-mRNA Splicing. Methods Mol Biol 1332, 3-23; Keck et al., 1989, Vascular permeability factor, an endothelial cell mitogen related to PDGF. Science 246, 1309-1312; Leung et al., 1989, Vascular endothelial growth factor is a secreted angiogenic mitogen. Science 246, 1306-1309; Plouet et al., 1989, Isolation and characterization of a newly identified endothelial cell mitogen produced by AtT-20 cells. EMBO J 8, 3801-3806). VEGF stimulates angiogenesis and vascular permeability by activating two tyrosine-kinase receptors, VEGFR1/Flt1 and VEGFR2/KDR (Shibuya and Claesson-Welsh, 2006, Signal transduction by VEGF receptors in regulation of angiogenesis and lymphangiogenesis. Exp Cell Res 312, 549-560).

The VEGF/VEGFRs pathway is a key mediator in the aggressiveness of clear cell renal cell carcinoma (ccRCC), the most frequent subtype of RCC (Escudier et al., 2019, Renal cell carcinoma: ESMO Clinical Practice Guidelines for diagnosis, treatment and follow-up. Ann Oncol 30, 706-720). The von Hippel-Lindau (VHL) tumor suppressor gene is inactivated in 80% of ccRCC leading to stabilization of the Hypoxia Inducible Factor 1 and 2 alpha and the subsequent overexpression of VEGF (Hsieh et al., 2017, Renal cell carcinoma. Nat Rev Dis Primers 3, 17009).

The treatment of ccRCC depends on the disease stage. Surgery is the standard of care for non-metastatic patients and adjuvant therapy is relevant only for patients with local invasion. In the metastatic phase, ccRCC is unfortunately refractory to conventional chemo/radiotherapy (Makhov et al., 2018, Resistance to Systemic Therapies in Clear Cell Renal Cell Carcinoma: Mechanisms and Management Strategies. Mol Cancer Ther 17, 1355-1364).

However, the hypervascularization context favored the use of anti-angiogenic therapies targeting VEGF or their receptors. Given the crucial nature of this pathway in tumorigenesis, signaling activation of VEGF-A has been the focus of investigation in the last decade. Several clinical trials demonstrated their efficiency in 2007 on progression-free survival as compared to the reference treatment at that time, interferon alpha.

Following completion of the clinical trials, the Food and Drug Administration (FDA) approved the small ATP mimetics sorafenib (Escudier et al., 2009, Sorafenib for treatment of renal cell carcinoma: Final efficacy and safety results of the phase III treatment approaches in renal cancer global evaluation trial. J Clin Oncol 27, 3312-3318) and sunitinib (Motzer et al., 2009, Overall survival and updated results for sunitinib compared with interferon alfa in patients with metastatic renal cell carcinoma. J Clin Oncol 27, 3584-3590) for the treatment of metastatic ccRCC.

The FDA also approved bevacizumab, an anti-VEGF monoclonal antibody, for the treatment of metastatic ccRCC in the first line in combination with interferon alpha (Escudier et al., 2010, Phase Ill trial of bevacizumab plus interferon alfa-2a in patients with metastatic renal cell carcinoma (AVOREN): final analysis of overall survival. J Clin Oncol 28, 2144-2150). Considering the major role played by tumor neovascularization, bevacizumab was also approved the treatment of metastatic colon (Hurwitz et al., 2004, Bevacizumab plus irinotecan, fluorouracil, and leucovorin for metastatic colorectal cancer. N Engl J Med 350, 2335-2342), non-small cell lung (Sandler et al., 2006, Paclitaxel-carboplatin alone or with bevacizumab for non-small-cell lung cancer. N Engl J Med 355, 2542-2550), breast (Miller et al., 2007, Paclitaxel plus bevacizumab versus paclitaxel alone for metastatic breast cancer. N Engl J Med 357, 2666-2676), and ovarian (Burger et al., 2011, Incorporation of bevacizumab in the primary treatment of ovarian cancer. N Engl J Med 365, 2473-2483) cancers in combination with standard chemotherapy.

Anti-VEGF-A therapies are important in the treatment of several cancers or neovascular pathologies such as age-related macular degeneration (AMD). The anti-VEGF-A agents include:
bevacizumab commercialized under the brand name AVASTIN® by Genentech,
ranibizumab commercialized under the brand name LUCENTIS® by Novartis and Genentech,
pegaptanib sodium (commercialized under the brand name MACUGEN® by Eyetech Pharmaceuticals and Pfizer, and
aflibercept commercialized under the brand name EYLEA® (VEGF Trap-Eye) by Regeneron Pharmaceuticals and Bayer.

Despite the combination treatment bevacizumab with chemotherapy increased the progression-free survival (PFS), its limited impact on overall survival (OS) resulted in the loose of FDA approval for breast (Sasich and Sukkari, 2012, The US FDAs withdrawal of the breast cancer indication for Avastin (bevacizumab), Saudi Pharm J 20, 381-385).

Bevacizumab combined with interferon lost its FDA approval in 2016 but was recently approved in combination with the anti-PDL1 antibody atezolizumab (Rini et al., 2019, Atezolizumab plus bevacizumab versus sunitinib in patients with previously untreated metastatic renal cell carcinoma (IMmotion151): a multicenter, open-label, phase 3, randomised controlled trial. Lancet).

Although inhibitors of signaling activation of VEGF-A are successfully used in the clinic, not all patients respond to the treatment and some patients fail to fully respond to angiogenesis inhibitor therapy.

The complexity of VEGF biology could in part explain such limited efficacy as compared to the multi spectrum of tyrosine kinase inhibitors targets. The VEGF is regulated during all the processes of its expression including transcription of its gene, splicing of its pre-mRNA, stabilization, destabilization of its mRNA and translation (Apte et al., 2019, VEGF in Signaling and Disease: Beyond Discovery and Development. Cell 176, 1248-1264).

Alternative splicing of VEGF pre-mRNA generates mRNAs coding for pro-angiogenic isoforms known as VEGFₓₓₓ (VEGF₁₂₁, VEGF₁₆₅, VEGF₁₈₉ and VEGF₂₀₆, XXX corresponding to the number of aminoacid minus the signal peptide of each isoforms).

In 2002, an alternative 3′ splice site was discovered in exon 8 of the human VEGF gene, creating the VEGF_{xxxb} family. VEGF_{xxxb} isoforms differ from the VEGFₓₓₓ in the last six amino acids (CDKPRR SEQ ID NO: 9 for VEGFₓₓₓ, SLTRKD SEQ ID NO:10 for VEGF_{xxxb}) (Harper and Bates, 2008, VEGF-A splicing: the key to anti-angiogenic therapeutics? Nat Rev Cancer 8, 880-887).

While VEGFₓₓₓ isoforms have pro-angiogenic, pro-permeability and pro-migratory properties, VEGF_{xxxb} isoforms exert less potent effect on these parameters and were considered as anti-angiogenic.

The same controversy was described for VEGF-Ax which results from a translational readthrough the stop codon generating a longer VEGF isoform (Eswarappa et al., 2014, Programmed translational readthrough generates antiangiogenic VEGF-Ax. Cell 157, 1605-1618; Xin et al., 2016, Evidence for Pro-angiogenic Functions of VEGF-Ax. Cell 167, 275-284 e276).

Accordingly, there is still a need for improved angiogenesis inhibitor and/or vasculogenesis inhibitor therapy.

### Problem to be Solved

The technical problem underlying the present invention is thus the provision of improved or alternative means and methods for the treatment of angiogenic diseases.

Since the discovery of VEGF in 1989, none of the discovered isoforms could explain the complexity of VEGF biology and its limited efficacy in some specific treatments.

All the splices described to date moderately affect the general sequence with insertion of six amino acids for the alternative eighth exons 8a or 8b, twelve amino acids for exon 7b, seventeen amino acids for exon 6b, twenty-five amino acids for exon 6a and thirty-two amino acids for exon 7a (Guyot and Pagès, 2015, VEGF Splicing and the Role of VEGF Splice Variants: From Physiological-Pathological Conditions to Specific Pre-mRNA Splicing. Methods Mol Biol 1332, 3-23).

Modification of the C-terminal part of the protein replacing the NRP (Neuropilin) binding domain of conventional VEGF by an alternative sequence was already described for the VEGFxxxb isoforms in which the CDKPRR sequence, the NRP1 binding domain, was modified to SLTRKD (Harper and Bates, 2008, VEGF-A splicing: the key to anti-angiogenic therapeutics? Nat Rev Cancer 8, 880-887). Modification of this C-terminal part was also described for VEGF-Ax, a form of VEGF resulting from translation throughout the stop codon (Eswarappa et al., 2014, Programmed translational readthrough generates antiangiogenic VEGF-Ax. Cell 157, 1605-1618). Anti-angiogenic properties were first described for the VEGFxxxb and VEGF-Ax isoforms. Less potent as compared to VEGF, pro-angiogenic properties were also associated to both isoforms (Catena et al., 2010, VEGF (1) (2) (1) b and VEGF (1) (6) (5) b are weakly angiogenic isoforms of VEGF-A. Mol Cancer 9, 320; Xin et al., 2016, Evidence for Pro-angiogenic Functions of VEGF-Ax. Cell 167, 275-284 e276).

The technical problem is solved by provision of the embodiments provided herein below and as characterized in the appended claims.

The present invention is based on the discovery by the inventors of the existence of a new alternative splice acceptor site in the seventh intron. According to the known results, the inventors expected that the modification of the C-terminal part in the new splice variant isoform of VEGF should result in the same controversy. However, unexpectedly, the resulting new alternative splicing leads to the production of a new isolated splice variant isoform of VEGF displaying physiological pro-angiogenic, pro-lymphangiogenic, pro-permeability and pro-migratory properties.

The existence of this biological different isoform revisited the VEGF field and suggests that VEGF secrets can be highlighted thirty years after its discovery. The results of the invention constitute an important breakthrough in the field of angiogenesis and explain major failures of anti-VEGF therapies. Considering the new isoform of VEGF according to the invention appears to be at the origin of new therapeutic strategies for several pathologies in which the VEGF/VEGF_{NF}/angiogenesis axis is a key driver.

### SUMMARY OF THE INVENTION

A first object of the invention is to provide a new isolated splice variant isoform of VEGF having pro-angiogenic and pro-lymphangiogenic activity, said isoform comprising an amino acid sequence having at least 80%, preferably at least 95%, identity to the amino acid sequence of SEQ ID NO:1.

A second object is to provide both an isolated cDNA nucleotide molecule capable of encoding the isolated splice variant isoform of VEGF according to claim 1, said cDNA molecule comprising a nucleotide sequence having at least 80%, preferably at least 95%, identity to the nucleotide sequence of SEQ ID NO:3 and an isolated RNA nucleotide molecule having a sequence which is transcribed from the cDNA according to claim 5, said RNA molecule comprising a nucleotide sequence having at least 80%, preferably at least 95%, identity to the nucleotide sequence of SEQ ID NO:4.

A further object of the invention is the isolated isoform of VEGF according to the invention or the isolated nucleotide molecule according to the invention, for use as an active pharmaceutical substance.

Another object is an inhibitor of the pro-angiogenic and pro-lymphangiogenic activity of the isolated isoform of VEGF according to the invention or the isolated nucleotide molecule according to the invention for use as an active pharmaceutical substance.

Another object is the isolated isoform of VEGF according to the invention for use as a prognostic marker and as a predictive marker of the efficacy of specific treatments.

Herein is also disclosed the use of the isolated isoform of VEGF according to the invention or the isolated nucleotide molecule according to the invention as an immunogen to produce an antibody immunospecific for such isolated isoform, preferably for VEGF_{222/NF}, or nucleotide sequences respectively, an antibody raised against the isolated isoform of VEGF according to the invention, a process inhibiting or favoring splicing towards this isoform, an expression vector comprising the sequence of a nucleotide molecule according to the invention, a host cell comprising an expression vector according to the invention, a method of screening compounds to identify an inhibitor of the pro-angiogenic and lymphangiogenic activity of the isolated isoform of VEGF according to the invention and an assay for the specific detection of the isolated isoform VEGF_{222/NF} according to the invention in a sample comprising carrying out a polymerase chain reaction on at least a portion of the sample using the following primer sequences: Forward primer of SEQ ID NO:7 and Reverse primer of SEQ ID NO:8.

Further aspects and advantages of the present invention are described in the following description (with reference to FIGS. 1 to 14), which should be regarded as illustrative and not limiting the scope of the present application.

### BRIEF DESCRIPTION OF THE DRAWINGS

FIG. 1. A) a DNA sequence coding for the VEGF_{222/NF} protein produced by ProteoGenix (SEQ ID NO:69) with B) the corresponding protein sequence produced by ProteoGenix (SEQ ID NO:2).

FIG. 2. Peptides #1 (SEQ ID NO:5) and #2 (SEQ ID NO:6) specific of the VEGF_{222/NF} and used for rabbit immunization to produce polyclonal antibodies by ProteoGenix.

FIG. 3. The new VEGF splicing variant VEGF_{222/NF} encodes a protein conserved between species and expressed in normal tissues and in cancer cells. A) Splicing possible events of the VEGF pre-mRNA (SEQ ID NO:11) and the resulting C-terminal specific sequence of the VEGF_{222/NF} and VEGF₁₆₅ (SEQ ID NO:12). A′) VEGF_{222/NF} splice variants. The primers used for RT-(q)PCR analyzes are indicated. B) Conservation of C-terminal sequence of VEGF_{222/NF} between species. HS: Homo Sapiens; GG: Gorilla Gorilla; PT: Pan Troglodytes; SS: Sus Scrofa; CL: Canis Lupus; MM: Mus Musculus; RN: Rattus Norvegicus. C) RT-PCR analysis of the expression of VEGF_{222/NF} and VEGF in RCC cell lines and non-tumoral kidney sample. D) RT-PCR analysis of the expression of VEGF_{222/NF} and VEGF in non-tumoral human tissues samples. E) RT-qPCR analysis of the expression of VEGF_{222/NF} and VEGF in RCC cell lines. ***P<0.001 vs VEGF in TIME; ###P<0.001 vs VEGF₂₂₂NF in TIME. F) Assessment of VEGF and VEGF_{222/NF} expression in breast cancer (MDA-MB-231), medulloblastoma (DAOY) and pancreatic ductal adenocarcinoma (MiaPaca-2) cells by RT-qPCR. ***P<0.001 vs VEGF in TIME, ###P<0.001 vs VEGF_{222/NF} in TIME. G) ELISA analysis of the expression of VEGF_{222/NF} and VEGF in the supernatant of RCC cell lines. ***P<0.001 vs VEGF in TIME; ###P<0.001 vs VEGF_{222/NF} in RCC10. H) RT-PCR analysis of the expression of the different VEGF_{222/NF} and VEGF isoforms in 786-O and ACHN cells. I) RT-PCR analyzes of VEGF/NF and VEGF expression in non-tumor human tissue samples

FIG. 4. Characterization of anti-VEGF_{222/NF} antibodies. A) Validation of specific anti-VEGF_{222/NF} antibodies. Two antibodies targeting the epitope 1 and two antibodies targeting the epitope 2 were evaluated. Samples: 1) GST-NF, 2) Empty vector (EV), 3) pcDNA3.1-VEGF_{222/NF} were loaded on an acrylamide gel and immuno blots were performed using the four different antibodies and the rabbit pre-immune serum. B) Specificity of anti-VEGF_{222/NF} antibodies. 5 ng (rVA) or 20 ng (rVA′) of recombinant VEGF₁₆₅ or 5 ng (rVB) or 20 ng (rVB′) of recombinant VEGF_{165b}, or conditioned media of HEK293 cells transfected with empty vector (EV), a vector coding for VEGF₁₆₅ (pL6VA), or two independent vectors coding for VEGF_{222/NF} (pCNF) or (pL6NF) were loaded on an acrylamide gel and immuno blots were performed using the anti-VEGF_{222/NF} #2.2 antibody. C) GST-222/NF protein, cell lysate or conditioned media of HEK293 cells expressing EV, pL6VA, pCNF, pL6NF were loaded on an acrylamide gel and immunoblot was performed using the anti-VEGF_{222/NF} #2.2 antibody or anti-HSP90 as a loading control.

FIG. 5. VEGF_{222/NF} binds VEGF-receptors and stimulates endothelial cell proliferation, migration, permeability and angiogenesis. Specific binding of VEGF_{222/NF} to VEGFRs (VEGFR1, VEGFR2, VEGFR3) A) and to NRPs (NRP1, NRP2) A′).

A″) VEGF_{222/NF} induces phosphorylation of VEGFR2 and activation of the downstream signaling pathways AKT and ERK. Confluent monolayers of TIME cells were serum-starved for 2 h and then treated for the indicated times with VEGF₁₆₅ (100 ng/ml) or VEGF_{222/NF} (100 ng/ml). Cells were washed with PBS and lyzed with Laemmli buffer. Immuno blots were performed with the indicated antibodies. B) Cell proliferation assay of serum-starved endothelial cells (TIME) treated with VEGF₁₆₅ (100 ng/mL) or VEGF_{222/NF} (100 ng/ml). Cells were counted for 7 days. C) Wound scratch assays performed in serum-starved TIME cells treated with VEGF₁₆₅ (100 ng/ml) or VEGF_{222/NF} (100 ng/ml). D) Wound closure was determined at 3 h, 6 h, 9 h and 12 h following treatment. E) In vitro permeability assay. A monolayer of serum-starved TIME cells on 4-μm pore culture inserts were treated with VEGF₁₆₅ (100 ng/ml) or VEGF_{222/NF} (100 ng/ml) in the presence of AXITINIB (1 μM) for 30 min. Streptavidin-HRP was then added to the transwell for 10 minutes and TMB substrate was added in the lower compartment to assess permeability. F) In vivo permeability assay. Mice were injected with Evans blue dye intravenously, followed by PBS, VEGF₁₆₅ (500 ng/ml) or VEGF_{222/NF} (500 ng/ml) in the ears. After 20 minutes, ears were recovered, and the amount of Evans blue was determined by colorimetry (top panel). Representative photographs of the vascular leakage induced by PBS, VEGF₁₆₅ or VEGF_{222/NF} (bottom panel). G) In vivo plug assay. Mice were injected with low concentration MATRIGEL® containing PBS, VEGF₁₆₅ (1 Mg/ml) or VEGF_{222/NF} (1 kg/mL) and hemoglobin was measured 15 days after implantation. Representative photographs of the MATRIGEL® plug 15 days after implantation (bottom panel). Data were expressed as mean±S.E.M. **P<0.01 vs PBS, ***P<0.001 vs PBS, #P<0.05. ##P<0.01 vs VEGF₁₆₅.

FIG. 6. VEGF_{222/NF} promotes the proliferation and the survival of RCC cells. A) RT-qPCR analysis of the expression of VEGF_{222/NF} and VEGF in ACHN-overexpressing VEGF₁₆₅ or VEGF_{222/NF}. ACHN cells were transduced with plenti6.3 expressing full-length VEGF₁₆₅ CDNA or VEGF_{222/NF} and VEGF₁₆₅ or VEGF_{222/NF} mRNA expression was assessed. *** P<0.001 vs LacZ. B) ELISA assay of VEGF_{222/NF} and VEGF in the supernatant of ACHN-overexpressing VEGF₁₆₅ or VEGF_{222/NF} cells. ***P<0.001 vs LacZ. C) Proliferation of ACHN-overexpressing VEGF₁₆₅ or VEGF_{222/NF}. Cells were counted for 7 days. ***P<0.001 vs LacZ. D-E) RT-qPCR analysis of the expression of VEGF_{222/NF} and VEGF in ACHN (D) and in 786-O (E) cells transduced with pLKO.1 expressing shVEGF₁₆₅ or shVEGF_{222/NF}. *P<0.05. **P<0.01, ***P<0.001 vs Scramble. F) Clonogenic assay assessed in ACHN-(top) and in 786-O-(bottom)-VEGF₁₆₅ or -VEGF_{222/NF} downregulated cells 7 days after transduction.

FIG. 7. VEGF_{222/NF} induces tumor cell proliferation through NRP1. A) mRNA expression of NRP1 and NRP2 of ACHN cells transfected with shScramble, shNRP1 or shNRP2. B-C) Cell proliferation assay of ACHN-VEGF₁₆₅ and ACHN-VEGF_{222/NF} cells transfected with shNRP2 (B) or shNRP1 (C). **P<0.01, ***P<0.001 vs shScramble.

FIG. 8. VEGF_{222/NF} stimulates human dermal lymphatic endothelial cells (HDLECs) proliferation and induces phosphorylation of VEGFR3. A) HDLECs cells (25.000) were seeded in 6-well plates in Endothelial Cell Growth Medium (Promocell) containing 0.5% FBS. Twenty-four hours later, cells were treated with VEGF₁₆₅ (100 ng/ml), VEGF_{222/NF} (100 ng/ml) or VEGFC (100 ng/ml) (Day 0) and were counted at 0, 24, 48 and 72 hours. Results were expressed as fold increase considering day 0 as the reference. *P<0.05, ** P<0.01, ***P<0.001 vs PBS, #P<0.05, ##P<0.01 vs VEGF₁₆₅, § P<0.05. §§ P<0.01 vs VEGF_{222/NF}. B) ELISA assay of VEGFR3 activation. Phospho-VEGFR3 (active VEGFR3) levels were measured by ELISA following starved-HDLECs exposure for 15 min to VEGF₁₆₅, VEGF_{222/NF} or VEGFC (100 ng/ml). Results are expressed as pg of phospho-VEGFR3/μg of proteins. **P<0.01 vs VEGF_{222/NF}. ND: No Detectable.

FIG. 9. VEGF_{222/NF} promotes tumor growth and induces tumor angiogenesis, lymphangiogenesis and vessel maturation. A) Tumor incidence determined in nude mice bearing ACHN-LacZ, ACHN-VEGF₁₆₅, ACHN-VEGF_{222/NF} (n=10 per group) tumors. B) Tumor growth curves of ACHN-LacZ, ACHN-VEGF₁₆₅, ACHN-VEGF_{222/NF}. Tumor volumes were measured each week with a caliper for 70 days. C) Relative hemoglobin content of ACHN-LacZ. ACHN-VEGF₁₆₅, ACHN-VEGF_{222/NF} tumors. D) Representative photographs of the tumors ACHN-LacZ, ACHN-VEGF₁₆₅, ACHN-VEGF_{222/NF} displaying blood vessels (top panel) and lymphatic vessels (indicated by the black stars) (bottom panel). E) Average diameter of peri-tumoral vessels of the first (<140 μm), second (between 140 and 213 μm diameter) and third quartile (>213 μm). F) RT-qPCR analysis of angiogenic and lymphangiogenic genes expressed in ACHN-LacZ, ACHN-VEGF₁₆₅, ACHN-VEGF_{222/NF} tumors. G) Immunofluorescence detection of CD31 and a-SMA (top panel) and LYVE1 (bottom panel) in ACHN-LacZ, ACHN-VEGF₁₆₅, ACHN-VEGF_{222/NF} tumors. H) Number of immature (CD31+) and mature (CD31+, a-SMA+) vessels in ACHN-LacZ, ACHN-VEGF₁₆₅. ACHN-VEGF_{222/NF} tumor sections. I) Number of lymphatic vessels (LYVE1+) in ACHN-LacZ, ACHN-VEGF₁₆₅, and ACHN-VEGF_{222/NF} tumor sections. *P<0.05, **P<0.01 vs LacZ, *** P<0.001 vs LacZ. #P<0.05. ##P<0.01 vs VEGF₁₆₅.

FIG. 10. VEGF_{222/NF} inhibition delays tumor growth of experimental RCC. 3M of 786-O cells were subcutaneously grafted in the left flank of NMRI mice. Once tumors reached 80 mm³, mice were treated by intraperitoneal injection of control or anti-VEGF_{222/NF} antibodies (see materials and methods for the obtention of the antibodies), or bevacizumab once a week for 6 weeks (5 mg/kg). Tumor volumes were monitored with a caliper for 50 days. ***P<0.001, ****P<0.0001 (Anova Test).

FIG. 11. VEGF_{222/NF} is associated with metastatic dissemination in zebrafishes. A) Representative photographs of zebrafish embryos (n=35) injected with red-DID labelled ACHN-LacZ. ACHN-VEGF₁₆₅, and ACHN-VEGF_{222/NF} into the perivitelline space. Zebrafish embryos were monitored for tumor metastases using fluorescent microscope (centre and right panels). B) Table representing the number of zebrafish embryos with disseminated tumor foci in the tail. C) Area of metastasis in the zebrafish embryos tails were quantified at 24, 48 and 72 h following tumor injection. **P<0.01 vs LacZ, *** P<0.001 vs LacZ. ##P<0.01, ###P<0.001 vs VEGF₁₆₅.

FIG. 12. A) Bevacizumab has a lower affinity for VEGF_{222/NF} than for VEGF₁₆₅. Bevacizumab saturation binding experiments on VEGF₁₆₅ and VEGF_{222/NF}. Recombinant VEGF₁₆₅ and VEGF_{222/NF} proteins (100 ng/well) were incubated with serial dilutions of bevacizumab (10-2 to 107 pM) were incubated and specific binding was determined (GraphPad Prism, V8). Anti-VEGFXXX/NF antibodies specifically recognize VEGF_{222/NF}. (B) Immuno-blotting. Recombinant KLH, VEGF165 or VEGF_{222/NF} (100 ng) were loaded onto acrylamide gels. Proteins were identified using the mouse anti-VEGFXXX/NF (1/2000). (C) ELISA. KLH and VEGF_{222/NF} (100 ng/well) were immobilized overnight on 96-well plates and then incubated with the mouse anti-VEGFXXX/NF (1/2000). Detection was performed with TMB. Results are expressed as Optical Density (OD) values. **P<0.01 vs KLH.

(D) The growth curve of experimental tumors generated with 786-0 cells following anti-KLH (n=6), and anti-VEGF_{XXX/NF} (n=5) and bevacizumab (n=5) treatment. (E) The weight of 786-O tumors at the end of the experiment. (F) Quantification of Ki67 positive cells in 786-0 tumors. Cell proliferation was revealed by Ki67 immunofluorescent labeling and Hoechst33342 nuclear DNA counterstaining. (G) Number of mature (CD31+, a-SMA+) vessels in the different tumor sections. (H) Number of lymphatic vessels (LYVE1+) in the tumor sections. *P<0.05, ***P<0.001 vs control, #P<0.01, ##P<0.01, ###P<0.001 vs bevacizumab.

Plasma levels of VEGFXXX/NFand VEGF are increased in the VEGF bevacizumab-treated group. ELISA of plasma levels of (I) and VEGFXXX/NF (J) in 786-O tumor-bearing mice treated with bevacizumab or KLH or anti-VEGFXXX/NF antibodies. *P<0.05, **P<0.01 vs KLH, #P<0.05vs bevacizumab. FIG. 13. A-D) The levels of VEGF and VEGF_{222/NF} were evaluated in the plasma (just before sunitinib treatment) of 47 metastatic ccRCC patients. The third quartile was used as the cut-off to determine patients' groups, respectively 4500 μg/ml and 3000 μg/ml for VEGF and VEGF_{222/NF}. The plasma levels of VEGF (A and C) or VEGF_{222/NF} (B and D) were correlated to PFS under first-line sunitinib treatment (A and B) or with OS (C and D). Kaplan-Meier method was used to produce survival curves and analyzes of censored data were performed using Cox models. Statistical significance (p values) is indicated.

FIG. 14. Predictive value of VEGF and VEGF_{222/NF} co-detection in M1 ccRCC patients. VEGF_{222/NF} plasma levels were determined in metastatic ccRCC patients just-before sunitinib treatment.

FIG. 15. Primers used for qPCR and PCR.

### DETAILED DESCRIPTION

The present inventors have identified a new alternative splice acceptor site, notably present in the last intron of the VEGF pre-mRNA resulted in the insertion of 23 bases that shifted the open reading frame giving rise to a human VEGF isoform minus the signal peptide, of 222 amino acids. This novel isoform has been designated VEGF_{222/NF}. VEGF_{222/NF} stimulates endothelial cell proliferation and vascular permeability through VEGFR2 activation.

According to a first aspect of the invention, there is provided an isolated splice variant isoform of VEGF having pro-angiogenic and pro-lymphangiogenic activity, said isoform comprising an amino acid sequence having at least 80%, preferably at least 95%, identity to the amino acid sequence of SEQ ID NO:1, including certain pro-angiogenic and pro-lymphangiogenic variants thereof, as defined in and by the appended claims.

The term “isolated” as used herein means altered from its natural state, i.e. if it occurs in nature, it has been changed or removed from its original environment, or both. For example, a polynucleotide or a polypeptide naturally present in a living organism is not “isolated”, but the same polynucleotide or polypeptide separated from the coexisting materials of its natural state is “isolated”, as the term is used herein. Moreover, a polynucleotide or polypeptide that is introduced into an organism by transformation, genetic manipulation or by another recombinant method is “isolated” even if it is still present in said organism, which organism can be living or non-living.

The term “isoform of VEGF” means a polypeptide variant of VEGF. The term “polypeptide(s)” as used herein refers to any peptide or protein comprising two or more amino acids joined to each other by peptide bonds or modified peptide bonds. “Polypeptide(s)” refers to both short chains, commonly referred to as peptides, oligopeptides, and oligomers and to longer chains generally referred to as proteins. Polypeptides can contain amino acids other than the 20 gene encoded amino acids. “Polypeptide(s)” include those modified either by natural processes, such as processing and other post-translational modifications, but also by chemical modification techniques. Such modifications are well described in research literature, and are well known to those skilled in the art. It will be appreciated that the same type of modification can be present at the same or varying degrees at several sites in a given polypeptide. Also, a given polypeptide can contain many types of modification. Modification can occur anywhere in a polypeptide, including the peptide backbone, the amino acid side chains, and the amino or carboxyl termini. Modifications include, for example, acetylation, acylation, ADP-ribosylation, amidation, covalent attachment of flavin, covalent attachment of a heme moiety, covalent attachment of a nucleotide or a nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation or glutamic acid residues, hydroxylation and ADP-ribosylation, selenoylation, sulfation, transfer-RNA mediated addition of amino acids to proteins, such as arginylation, and ubiquitination. Polypeptides can be branched, or cyclic, with or without branching. Cyclic, branched, and non-branched polypeptides can result from post-translational natural processes and can be made by entirely synthetic methods as well.

The term “nucleotide(s)” as used herein generally refers to any polyribonucleotide or polydeoxyribonucleotide, which may be unmodified RNA or DNA or modified RNA or DNA. Polynucleotide(s) include, without limitation, single- and double-stranded DNA, DNA that is a mixture of single- and double-stranded regions or single- and triple-stranded regions, single- and double-stranded RNA, and RNA that is a mixture of single- and double-stranded regions, hybrid molecules comprising DNA and RNA that can be single-stranded or, more typically, double-stranded, or triple-stranded regions, or a mixture of single- and double-stranded regions. As used herein, the term “polynucleotide(s)” also includes DNAs or RNAs as described above that contain one or more modified bases. Thus, DNAs or RNAs with backbones modified for stability or for other reasons are “polynucleotide(s)” as the term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as used herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

The isoform of VEGF according to the invention comprises an amino acid sequence having at least 80%, e.g. 81%, 83%, 85%, 87%. 90%, 93%. 95%, 97%, 99%, preferably at least 95%, more preferably at least 99%, identity to the amino acid sequence of SEQ ID NO:1.

“Identity”, as used herein, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in the following references (Computational Molecular Biology, Lesk A. M., ed., Oxford University Press, New York, 1988; Biocomputing: Informatics and genome Projects, Smith D. W., ed., Academic Press, New York. 1993; Computer Analysis of sequence Data, Part I, Griffin A. M., and Griffin H. G., eds., Humana Press. New jersey, 1994; sequence Analysis in Molecular Biology, von Heinje G., Academic Press, 1987; and sequence Analysis Primer, Gribskov M. and Devereux J., eds., M Stockton Press, New York, 1991; and Carillo H., and Lipman D., SIAM J. Applied Math., 48:1073 (1998)). Methods to determine identity are designed to give the largest match between the sequences tested. Moreover, methods to determine identity are codified in publicly available computer programs. Computer program methods to determine identity between two sequences include, but are not limited to, the GCG program package (Devereux J. et al., Nucleic Acids Research 12 (1): 387 (1984)), BLASTP, BLASTN, and FASTA (Altschul S. F. et al., J. Molec. Biol. 215:403-410 (1990)). The BLAST X program is publicly available from NCBI and other sources (BLAST Manual, Altschul S. et al., NCBI NLM NUH Bethesda, MD 20894; Altschul S. et al., J. Mol Biol. 215:403-410 (1990)).

The term “variant(s)” as used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and/or truncations in the polypeptide encoded by the reference sequence, as discussed below. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more substitutions, additions, and/or deletions in any combination. A substituted or inserted amino acid residue may or may not be one encoded by the genetic code. The present invention also includes variants of each of the polypeptides of the invention, that is polypeptides that vary from the references by conservative amino acid substitutions, whereby a residue is substituted by another with like characteristics. Typical conservative amino acid substitutions are among Ala, Val, Leu and Ile; among Ser and Thr; among the acidic residues, Asp and Glu; among Asn and Gln; and among the basic residues Lys and Arg; or aromatic residues Phe and Tyr. Such conservative mutations include mutations that switch one amino acid for another within one of the following groups:
1. Small aliphatic, nonpolar or slightly polar residues: Ala, Ser, Thr, Pro and Gly;
2. Polar, negatively charged residues and their amides: Asp, Asn, Glu and Gln;
3. Polar, positively charged residues: His, Arg and Lys;
4. Large aliphatic, nonpolar residues: Met, Leu, Ile, Val and Cys; and
5. Aromatic residues: Phe, Tyr and Trp.

Such conservative variations can further include the following:

| Original Residue | Variation |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

Particularly preferred are variants in which several, e.g., 5-10, 1-5, 1-3, 1-2 or 1 amino acids are substituted, deleted, or added in any combination. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to a person skilled in the art.

Preferably, the isolated isoform of VEGF according to the invention comprises the amino acid sequence of SEQ ID NO:1.

More preferably, the isolated isoform of VEGF according to the invention is VEGF_{222/NF} and consists of SEQ ID NO:1.

More preferably, the isolated isoform of VEGF according to the invention consists of SEQ ID NO:2, an optimized sequence.

The new isoform VEGF_{222/NF} preferably described in the present invention inserted sixty-four additional amino acids. The insertion of the 23 bp (including AG) creates a new open reading frame allowing the translation to occur in the domain considered as the 3′ untranslated region (3′UTR) of the VEGF mRNA. The mRNA resulting from this alternative splicing, codes for a new human VEGF isoform of 248 amino acids from the initiation methionine. According to the international nomenclature, removal of the signal peptide gives rise to the VEGF_{222/NF} of 222 amino acids.

Additional splice acceptors sites are present in the different VEGF introns. 229 “AG” consensus sites are present only in the first intron, and several in the other introns which multiple the potential number of splice events in the VEGF gene. This possible multiplication of VEGF isoforms opens a new area of research in the VEGF field.

A further aspect of the invention provides an isolated cDNA nucleotide molecule capable of encoding the isolated splice variant isoform of VEGF according to the invention, said cDNA molecule comprising a nucleotide sequence having at least 80%, e.g. 81%, 83%, 85%, 87%, 90%, 93%, 95%, 97%, 99%, preferably at least 95%, more preferably at least 99%, identity to the nucleotide sequence of SEQ ID NO:3.

Preferably, the isolated cDNA nucleotide molecule comprises the nucleotide sequence of SEQ ID NO:3, more preferably consists of SEQ ID NO:3.

A further aspect of the invention provides an isolated RNA nucleotide molecule having a sequence which is transcribed from the cDNA according to the invention, said RNA molecule comprising a nucleotide sequence having at least 80%, e.g. 81%, 83%. 85%, 87%, 90%, 93%, 95%, 97%, 99%, preferably at least 95%, more preferably at least 99%, identity to the nucleotide sequence of SEQ ID NO:4.

Preferably, the isolated RNA nucleotide molecule comprises the nucleotide sequence of SEQ ID NO:4, more preferably consists of SEQ ID NO:4.

Preferably, the isolated isoform of VEGF according to the invention or the isolated nucleotide molecule according to the invention, derived from a mammalian sequence, wherein the mammalian sequence is selected from the group consisting of a primate, rodent, bovine or porcine sequence. More preferably, the sequence is derived from a human sequence.

A further aspect of the invention provides an expression vector comprising the sequence of a nucleotide molecule according to the invention, having at least 80%, preferably at least 95%, identity to the nucleotide sequence of SEQ ID NO:3, preferably the isolated cDNA nucleotide molecule comprises the nucleotide sequence of SEQ ID NO: 3, more preferably consists of SEQ ID NO:3.

A great variety of expression vector can be used to produce the new isoforms of VEGF according to the invention. Such vectors include, among others, chromosomal-, episomal- and viral-derived vectors, for example, vectors derived from plasmids, from bacteriophage, from transposons, from yeast episomes, from insertion elements, from yeast chromosomal elements, from viruses such as baculoviruses, papova viruses, such as SV40, vaccinia viruses, adenoviruses, adeno-associated viruses, fowl pox viruses, pseudorabies viruses, picornaviruses and retroviruses, and vectors derived from combinations thereof, such as those derived from plasmid and bacteriophage genetic elements, such as cosmids and phagemids. The expression vector constructs can contain control regions that regulate as well as engender expression. Generally, any system or vector suitable to maintain, propagate or express polynucleotides or to express a polypeptide in a host can be used for expression in this regard. The appropriate DNA sequence can be inserted into the expression vector by any of a variety of well-known and routine techniques, such as, for example, those set forth in Sambrook et al. (MOLECULAR CLONING: A LABORATORY MANUAL (supra)).

A further aspect of the invention provides a host cell comprising an expression vector according to the invention.

For recombinant production of the new isoforms of VEGF according to the invention, host cells can be genetically engineered to incorporate expression vectors or portions thereof or isoforms of the invention. Introduction of a polynucleotide into a host cell may be realized by methods described in many standard laboratory manuals and in publications such as Wang T Y et al. (Expression vector cassette engineering for recombinant therapeutic production in mammalian cell systems, Appl Microbiol Biotechnol. 2020 July; 104 (13): 5673-5688), such as, calcium phosphate transfection, DEAE-dextran mediated transfection, transfection, microinjection, cationic lipid-mediated transfection, electroporation, transduction, scrape loading, ballistic introduction and infection.

Representative examples of appropriate hosts include bacterial cells, such as streptococci, staphylococci, enterococci, coli, streptomyces, cyanobacteria, Bacillus subtilis; fungal cells, such as yeast, Kluveromyces, Saccharomyces, a basidiomycete, Candida albicans and Aspergillus; insect cells such as Drosophila S2 and Spodoptera Sf9; animal cells such as CHO, COS, Hela, C127, 3T3, BHK, 293, CV-1 and Bowes melanoma cells; and plant cells.

Isoforms of VEGF according to the invention can be recovered and purified from recombinant cell cultures by well-known methods, including ammonium sulphate or ethanol precipitation, extraction such as acid extraction, anion or cation exchange chromatography, gel filtration, phosphocellulose chromatography, hydrophobic interaction chromatography, affinity chromatography, hydroxylapatite chromatography, lectin chromatography, preparative electrophoresis, FPLC (Pharmacia, Uppsala, Sweden), HPLC (e.g., using gel filtration, reverse-phase or mildly hydrophobic columns). Most preferably, high performance liquid chromatography is employed for purification. Well known techniques for refolding proteins can be employed to regenerate an active conformation after denaturation of the polypeptide during isolation and/or purification. In vitro activity assays for isoforms of VEGF according to the present invention include, permeability assays in mouse ear, tyrosine kinase receptor activation assays, endothelial cell proliferation (e.g. thymidine incorporation, cell number or BrDU incorporation), cell migration assays (including scratch assays), tube formation, gel invasion assays or pressure or wire myograph assays. In vivo assays include angiogenesis assays using rabbit corneal eye pocket, chick chorioallantoic membrane assays, dorsal skinfold chamber assays, functional blood vessel density, blood flow, blood vessel number, tumor implantation assays (syngenic or heterogenic), tumor growth or vessel density assays, growth factor induced assays in hamster cheek pouch, rat, mouse or hamster mesentery, or sponge implant assay (Angiogenesis protocols-Ed. J. Clifford Murray; Humana Press, Totowa, New Jersey; ISBN 0-89603-698-7 (part of a Methods in Molecular Medicine series)).

A further aspect of the invention provides an isolated isoform of VEGF or an isolated nucleotide molecule according to preceding aspects of the present invention for use as an active pharmaceutical substance.

The isolated isoform of VEGF or the isolated nucleotide molecule according to the invention is preferably used for its pro-angiogenic and pro-lymphangiogenic activity to alleviate a symptom of a disease or disorder of the nervous system chosen from neurodegenerative disorders, neural stem cell disorders, neural progenitor disorders, ischemic disorders, neurological traumas, affective disorders, neuropsychiatric disorders, learning and memory disorders, Parkinson's disease and Parkinsonian disorders, Huntington's disease, Alzheimer's disease, Amyotrophic Lateral Sclerosis, spinal ischemia, ischemic stroke, spinal cord injury, cancer-related brain/spinal cord injury, schizophrenia and other psychoses, depression, bipolar depression/disorder, anxiety syndromes/disorders, phobias, stress and related syndromes, cognitive function disorders, aggression, drug and alcohol abuse, obsessive compulsive behavior syndromes, seasonal mood disorder, borderline personality disorder, cerebral palsy, life style drug, multi-infarct dementia, Lewy body dementia, age related/geriatric dementia, epilepsy and injury related to epilepsy, spinal cord injury, brain injury, trauma related brain/spinal cord injury, anti-cancer treatment related brain/spinal cord tissue injury, infection and inflammation related brain/spinal cord injury, environmental toxin related brain/spinal cord injury, multiple sclerosis, autism, attention deficit disorders, narcolepsy and sleep disorders, to stimulate the development of collateral circulation in cases of arterial and/or venous obstruction selected from myocardial infarcts, ischemic limbs, deep venous thrombosis, and/or postpartum vascular problems and to treat lymphedema post radiotherapy or Milroy disease in which the lymphatic vessel system is damaged, more preferably ischemic disorders, myocardial infarcts and lymphoedema related diseases chosen from neurodegenerative disorders, ischemic disorders, neurological traumas, Alzheimer's disease, Amyotrophic Lateral Sclerosis, spinal ischemia, ischemic stroke, spinal cord injury, cancer-related brain/spinal cord injury, multi-infarct dementia, spinal cord injury, brain injury, trauma related brain/spinal cord injury, and anti-cancer treatment related brain/spinal cord tissue injury.

The invention also enables a method for treating a mammalian patient, preferably a human, for diseases such as previously mentioned requiring pro-angiogenic and pro-lymphangiogenic activity, comprising supplying to the patient the isolated isoform of VEGF or the isolated nucleotide molecule for use according to preceding aspects of the invention.

A further aspect of the invention provides a pharmaceutical composition comprising the isolated isoform of VEGF or the isolated nucleotide molecule for use according to preceding aspects of the invention such as previously mentioned, and a pharmaceutically acceptable medium.

A pharmaceutically acceptable medium includes any, and all solvents, dispersion media, coatings, antibacterial and antifungal agents, isotonic and absorption delaying agents and the like suitable for administration to a mammalian host. The use of such media and agents for pharmaceutical active substances is well known in the art. Supplementary active ingredients can also be incorporated into the medicament of the present invention.

A further aspect of the invention provides an inhibitor of the pro-angiogenic and pro-lymphangiogenic activity of the isolated isoform of VEGF or the isolated nucleotide molecule according to preceding aspects of the invention for use as an active pharmaceutical substance, preferably for use in the prevention and the treatment of an angiogenesis-dependent disease condition.

Preferably, the inhibitor is chosen from an antibody, a protein, a siRNA or a shRNA, a CRISPR guide, or an antisense oligonucleotide.

Preferably, the angiogenesis-dependent disease is selected from the group of pathologies presenting exacerbated angiogenesis including tumor and metastasis, rheumatoid arthritis, atherosclerosis, neointimal hyperplasia, diabetic retinopathy and other complications of diabetes, trachoma, retrolental fibroplasia, neovascular glaucoma, age-related degeneration, macular diabetes, retinopathies, haemangiomas, immune rejection of transplanted corneal tissue, corneal angiogenesis associated with ocular injury or infection, vascular disease, obesity, psoriasis, arthritis, and gingival hypertrophy, more preferably hyper-vascularized cancers and eye disorders with exacerbated angiogenesis.

In a preferred embodiment, VEGF_{222/NF} exhibited reduced ability to stimulate endothelial cell proliferation which was consistent with the absence of the domain enabling NRP1 binding and the delayed VEGFR2 activation. However, the resulting blood vessels in experimental tumors resembled normal and functional ones covered with pericytes. This property of VEGF_{222/NF} favors tumor vascularization and promotes tumors growth. Hence, as the tumor progress, the VEGF_{222/NF}-dependent functional blood vessel network becomes a key actor of tumor cell dissemination. Unexpectedly. VEGF_{222/NF} also promotes the development of a lymphatic network which also favors metastatic spreading as highlighted in the zebrafish model. Thus, in advanced stages, VEGF_{222/NF} favors tumor aggressiveness. As for VEGF, VEGF_{222/NF} exerts its detrimental effects by promoting tumor vascularization but also by stimulating tumor cell proliferation through autocrine loops. Although several tumor cells co-express VEGF and their receptors VEGFR1-3 (Lee et al., 2007. Autocrine VEGF signaling is required for vascular homeostasis, Cell 130, 691-703), ccRCC cells do not express VEGFRs (Cao et al., 2008, Neuropilin-1 upholds dedifferentiation and propagation phenotypes of renal cell carcinoma cells by activating Akt and sonic hedgehog axes. Cancer Res 68, 8667-8672). Instead, they express NRP1 and NRP2 that mediate autocrine proliferation loops with VEGF and VEGFC (Cao et al., 2013, Neuropilin-2 promotes extravasation and metastasis by interacting with endothelial alpha5 integrin. Cancer Res 73, 4579-4590; Cao et al., 2008, Neuropilin-1 upholds dedifferentiation and propagation phenotypes of renal cell carcinoma cells by activating Akt and sonic hedgehog axes. Cancer Res 68, 8667-8672). NRP1, but not NRP2, represents an interesting signaling partner of VEGF_{222/NF} since its down-regulation lowers VEGF_{222/NF}-dependent proliferation. The CDKPRR motif is absent in the C-terminal part of the VEGF_{222/NF} sequence. However, a PGRRK motif is strongly conserved between species. A new basic rich domain could be generated by proteolytic cleavage enabling NRP1 binding.

More importantly, cells overexpressing VEGF_{222/NF} became addicted to this autocrine loop that exerts proliferation but also pro-survival properties. VEGF was also described as a driver of immune tolerance by stimulating the expression of immune checkpoints at the surface of T cells through the stimulation of VEGFR2 (Voron et al., 2015. VEGF-A modulates expression of inhibitory checkpoints on CD8+ T cells in tumors. J Exp Med 212, 139-148). It is thus possible to guess that VEGF_{222/NF} will have equivalent effects since it stimulates VEGFR2. Therefore, the inventors expect that targeting VEGF_{222/NF} should inhibit three major hallmarks of cancer: tumor cell proliferation, angiogenesis, and immune tolerance in advanced/metastatic stage of tumor development. The results clearly involved VEGFR2 and NRP1 in the VEGF_{222/NF}-dependent signaling pathway. Moreover, the inventors clearly showed that overexpression of VEGF_{222/NF} in tumors stimulates the development of a lymphatic network. In vitro experiments demonstrated that VEGF_{222/NF} exerts a direct effect on lymphatic endothelial cells through VEGFR3 activation. To the inventors' knowledge, this is the first VEGF isoform that activates lymphangiogenesis through this receptor.

In the present application, the inventors only addressed the VEGF-dependent neoplasms. However, VEGF is also involved in several pathologies especially eye diseases including vascular age-related macular degeneration (vAMD) for which anti-VEGF is the standard of care (Rosenfeld et al., 2006, Ranibizumab for neovascular age-related macular degeneration. N Engl J Med 355, 1419-1431). In this pathology, anti-VEGF are inefficient or transiently efficient in more than 30% of the patients who become blind two years after relapses. As for cancers, it is possible to guess that the presence of VEGF_{222/NF} would limit the therapeutic effect of the anti-VEGF.

High VEGF levels were detected in patients with COVID-19 (Ackermann et al., 2020, Pulmonary Vascular Endothelialitis, Thrombosis, and Angiogenesis in Covid-19. N Engl J Med 383, 120-128). They presented severe endothelial injuries in the lungs, alveolar damages with infiltration of perivascular lymphocytes. The presence of high VEGF_{222/NF} in the lungs suggest that this new form may play a key role in severely infected patients.

A further aspect of the invention provides a pharmaceutical composition comprising the inhibitor of the isolated isoform of VEGF or the isolated nucleotide molecule for use according to preceding aspects of the invention such as previously mentioned, and a pharmaceutically acceptable medium.

The invention enables a method for treating a mammalian patient, preferably a human, for diseases such as previously mentioned requiring anti-angiogenic and anti-lymphangiogenic activity, comprising supplying to the patient the inhibitor of the isolated isoform of VEGF or the isolated nucleotide molecule for use according to preceding aspects of the invention.

A further aspect of the invention provides the isolated isoform of VEGF according to preceding aspects of the invention for use as a prognostic marker and as a predictive marker of the efficacy of specific treatments.

Preferably, the isolated isoform of VEGF according to the invention is used to prognose non metastatic clear cell Renal Cell Carcinoma (ccRCC) in mammalian patients, preferably human patients.

Preferably, the isolated isoform of VEGF according to the invention is used to predict the efficacy of treatments by the compounds chosen from bevacizumab, sunitinib, ranibizumab, pegaptanib sodium, aflibercept, brolucizumab.

In a preferred embodiment, the presence of VEGF_{222/NF} was correlated to a poor prognosis in metastatic ccRCC which is consistent with the VEGF_{222/NF} effects in experimental tumors. The generalization of this concept to several tumors is now possible thank to the availability of the home-made ELISA assay. Bevacizumab, the anti-VEGF antibody, failed in increasing the OS of ccRCC and breast cancer patients that resulted in the loose of FDA approval for both cancers. The presence VEGF_{222/NF} and classical VEGF represents a plausible explanation of bevacizumab failure in both cancers. The presence of VEGFxxxb lowers the efficacy of bevacizumab in colon cancer (Bates et al., 2012, Association between VEGF splice isoforms and progression-free survival in metastatic colorectal cancer patients treated with bevacizumab. Clin Cancer Res 18, 6384-6391). These results were attributed to the anti-angiogenic role of VEGFxxxb (Harper and Bates, 2008, VEGF-A splicing: the key to anti-angiogenic therapeutics? Nat Rev Cancer 8, 880-887). However, it was also possible that modification of the C-terminal part of VEGF alters the affinity for bevacizumab. The VEGF acts as a dimer involving the cysteine residue of the extreme C-terminal part “CDKPRR” which is lost in VEGF_{222/NF}. The modification in the dimer conformation induced by the absence of the disulfide bridge alters the three-dimensional structure and probably the recognition by the bevacizumab. The modification in the 3D conformation of VEGF_{222/NF} homodimers or of VEGF/VEGF_{222/NF} heterodimers does not favor an optimal recognition by the bevacizumab. Bevacizumab displays a 10-fold higher affinity for VEGF₁₆₅ as compared to VEGF_{222/NF} reinforcing this hypothesis. The presence of VEGF_{222/NF} must be paralleled to the definition of optimal bevacizumab doses in early phase trials. Before bevacizumab approval, different doses have been tested: 5, 7.5, 10 and 15 mg/kg. Depending on the cancer type, dependent dosages were approved to manage maximal therapeutic activity and limited toxicity. Hence, the 10 mg/kg dosage was approved in combination with interferon alpha for ccRCC (Escudier et al., 2007. Bevacizumab plus interferon alfa-2a for treatment of metastatic renal cell carcinoma: a randomised, double-blind phase III trial. Lancet 370, 2103-2111). If ignoring the toxic effect, higher concentrations of bevacizumab should have been more efficient by inhibiting at the same time VEGF and VEGF_{222/NF}. An equivalent situation stands for the use of gefitinib in lung cancer patients. The drug present activity only in patients with specific mutations in the EGF receptor with a daily 250 mg dose. Higher doses that inhibit both wild-type and mutated forms of EGF receptor cannot be administered because of toxic effects (Lynch et al., 2004, Activating mutations in the epidermal growth factor receptor underlying responsiveness of non-small-cell lung cancer to gefitinib. N Engl J Med 350, 2129-2139). Considering that VEGF_{222/NF} is detrimental in advanced stage of ccRCC, the development of a specific antibody deserves to be considered. The detection of VEGF and VEGF_{222/NF} in the blood would serve as a companion test for administration of anti-VEGF_{222/NF} alone or in combination with bevacizumab.

Its overexpression in kidney cancer (RCC) cells stimulated their proliferation whereas its downregulation induced their death. RCC cells overexpressing VEGF_{222/NF} generated aggressive experimental tumors. Such aggressiveness relies on the development of blood and lymphatic vessels. VEGF_{222/NF} overexpression in metastatic RCC patients was synonymous of poor prognosis. Moreover, VEGF_{222/NF} predicted the efficacy of anti-angiogenic drugs in RCC patients. The existence of VEGF_{222/NF} with more efficient pro-angiogenic/lymphangiogenic properties revisited the VEGF field.

The resulting new alternative splicing leads to the production of the VEGF_{222/NF}. VEGF_{222/NF} displayed physiological pro-angiogenic, pro-permeability and pro-migratory properties. Its expression was evidenced in several cancer cell lines including ccRCC. It stimulates tumor growth and metastatic dissemination through the development of mature blood and lymphatic vessel networks. Its expression is of good prognosis in non-metastatic ccRCC patients whereas it is of poor prognosis in metastatic ones.

A further aspect of the invention provides the use of the isolated isoform of VEGF or the isolated nucleotide molecule according to preceding aspects of the invention as an immunogen to produce an antibody immunospecific for such isolated isoform, preferably of VEGF_{222/NF}, or nucleotide sequences respectively.

A further aspect of the invention provides an antibody raised against the isolated isoform of VEGF according to the invention.

Preferably, the antibody is specific to the amino acid sequence of SEQ ID NO:1.

More preferably, the antibody is specific to the epitopes of SEQ ID NO:5 and/or SEQ ID NO:6.

According to a preferred embodiment, the antibody is not bevacizumab.

Antibodies generated against the polypeptides or polynucleotides of the invention can be obtained by administering the polypeptides or polynucleotides of the invention, or epitope-bearing fragments of either or both, analogues of either or both, or cells expressing either or both, to an animal, preferably a non-human, using routine protocols. For preparation of monoclonal antibodies, any technique known in the art that provides antibodies produced by continuous cell line cultures can be used. Examples include various techniques, such as those in Kohler, G. and Milstein, C. (Nature 256:495-497 (1975)); Kozbor et al. (Immunology Today 4:72 (1983)); Cole et al. (pg. 77-96 in MONOCOLONAL ANTIBODIES AND CANCER THERAPY, Alan R. Liss, Inc. (1985)).

Techniques for the production of single chain antibodies (U.S. Pat. No. 4,946,778) can be adapted to produce single chain antibodies to polypeptides or polynucleotides of this invention. Also, transgenic mice, or other organisms such as other mammals, can be used to express humanized antibodies immunospecific to the polypeptides or polynucleotides of the invention.

Alternatively, phage display technology can be utilized to select antibody genes with binding activities towards a polypeptide of the invention.

The above-described antibodies can be employed to isolate or to identify clones expressing the polypeptides or polynucleotides of the invention to purify the polypeptides or polynucleotides by, for example, affinity chromatography.

The polynucleotides, polypeptides and antibodies that bind to or interact with a polypeptide of the present invention can also be used to configure screening methods for detecting the effect of added compounds on the production of mRNA or polypeptide in cells. For example, an ELISA assay can be constructed for measuring secreted or cell associated levels of polypeptide using monoclonal and polyclonal antibodies by standard methods known in the art. This can be used to discover agents which can inhibit or enhance the production of polypeptide (also called antagonist or agonist, respectively) from suitably manipulated cells or tissues.

The invention also provides a method of screening compounds to identify an inhibitor of the pro-angiogenic and lymphangiogenic activity of the isolated isoform of VEGF according to the invention, preferably VEGF_{222/NF}, wherein said isolated isoform and a labelled ligand of said isolated isoform are incubated in the presence and the absence of a candidate compound, wherein decreased pro-angiogenic and/or lymphangiogenic activity of said isolated isoform in the presence of said candidate compound compared to the pro-angiogenic and/or lymphangiogenic activity in the absence of said compound indicates that the candidate compound is an inhibitor, the method comprising the steps of:
a) incubating the isolated isoform and the labelled ligand in the presence and absence of a candidate compound;
b) comparing the pro-angiogenic and/or lymphangiogenic activity of the isoform incubated with the candidate compound with the pro-angiogenic and/or lymphangiogenic activity of the isolated isoform incubated in the absence of the candidate compound;
wherein decreased pro-angiogenic and/or lymphangiogenic activity of the isolated isoform incubated in the presence of the candidate compound compared with the pro-angiogenic and/or lymphangiogenic activity of the isolated isoform in the absence of the candidate compound indicates that the candidate compound is an inhibitor.

The invention further provides an assay for the specific detection of the isolated isoform VEGF_{222/NF} in a sample comprising carrying out a polymerase chain reaction on at least a portion of the sample using the following primer sequences:
Forward primer of SEQ ID NO:7
Reverse primer of SEQ ID NO:8.

Embodiments of the present invention will now be described by way of the following examples.

### EXAMPLES

### Materials and Methods

### Cell Lines

Renal cell carcinoma cell lines ACHN, A498, CAKI-2, RCC4, 786-0 and TIME (Telomerase-immortalized microvascular endothelial) were obtained from ATCC®. RCC10 cells were a kind gift of W. H. Kaelin (Dana-Farber Cancer Institute, Boston, MA). 786-O expressing VHL were a gift from Dr. Nathalie Mazure (Bellot et al., 2009). HDLECS were obtained from Promocell. Tumor cell lines were cultured in DMEM, supplemented with 1 mM sodium pyruvate, 2 mM Glutamax, and 7.5% FBS. TIME cells were cultured in vascular cell basal medium (ATCC® PCS-100-030™) supplemented with microvascular endothelial cell growth kit (ATCC® PCS-100-041™). The final concentration of each component in complete TIME growth medium is as follows: 5 ng/ml VEGF, 5 ng/mL EGF, 5 ng/ml FGF, 5 ng/ml IGF-1, 10 mM L-glutamine, 0.75 units/mL Heparin sulfate, 1 μg/ml Hydrocortisone, 50 μg/ml Ascorbic acid, 2% FBS. HDLECS were cultured in microvascular endothelial cell growth medium kit classic (Pelobiotech) with 10 mM L-glutamin, 5 ng/ml EGF, 0.75 units/mL Heparin sulfate, 1 g/ml Hydrocortisone 2% FBS. All cell lines were cultured in 5% CO2 at 37° C.

### Animal Experiments

For in vivo permeability assay, 6-weeks old female BALB/cJRj mice were injected with Evans blue dye in the tail vein, followed by PBS, VEGF₁₆₅ (500 ng/ml) or VEGF₂₂/NF (500 ng/ml) in a 10 μL volume in the ears. For in vivo angiogenesis assay, PBS, VEGF₁₆₅ (1 μg/mL) or VEGF_{222/NF} (1 μg/mL) were mixed to MATRIGEL® Growth Factor reduced (200 μL) and injected subcutaneously into 6 weeks-old female NMRI mice. For ACHN tumor xenografts, ACHN overexpressing LacZ (ACHN-LacZ), VEGF₁₆₅ (ACHN-VEGF₁₆₅ₐ) or VEGF_{222/NF} (ACHN-VEGF_{222/NF}), cells were resuspended in a 50:50 PBS/Matrigel® Growth Factor reduced solution and injected subcutaneously into 6 weeks-old female NMRI mice.

For generating control or polyclonal anti-VEGF_{222/NF} antibodies, 6 weeks BALB/cJRj mice (n=10) were immunized either with KLH carrier protein or with specific peptides SEQ 1 and SEQ2 coupled to KLH as previously described (Guyot et al., 2017, Targeting the pro-angiogenic forms of VEGF or inhibiting their expression as anti-cancer strategies. Oncotarget 8, 9174-9188) IgG were purified of Protein G Sepharose column. All animal studies were approved in advance by the local animal care committee (Veterinary service and direction of sanitary and social action of Monaco, Dr H. Raps.)

### Clinical Details

Informed consent was obtained from all individual participants included in the study. All patients gave written consent for the use of tumor and blood samples for research. This study was conducted in accordance with the Declaration of Helsinki. Primary tumor samples of 93 non-metastatic (M0) ccRCC patients were obtained from the Rennes and Bordeaux University Hospitals (UroCCR group, NCT03293563). For metastatic patients, the population of the study included 47 ccRCC patients from the prospective SUVEGIL (NCT00943839) and TORAVA (NCT00619268) trials and from a retrospective cohort from Pavia (Italy).

### Protein Production

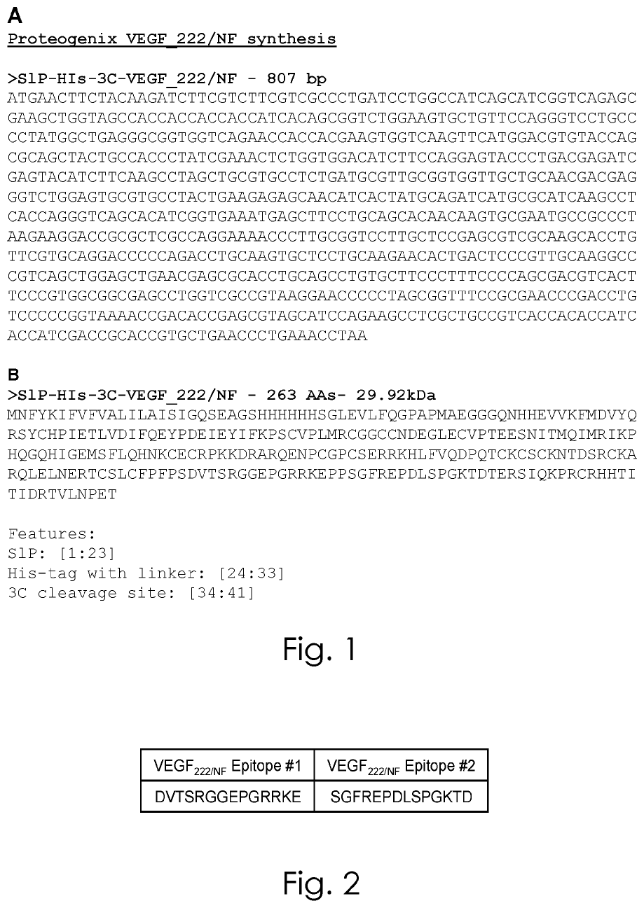

The recombinant VEGF_{222/NF} was produced in HEK293 mammalian cells by ProteoGenix (Schiltigheim, France). Briefly, the cDNA sequence was subcloned in ProteoGenix's proprietary mammalian cells expression vector pTXs2. The cDNA sequence is presented in the FIG. 1A. The construction was then transfected in HEK293 cells. VEGF_{222/NF} was purified using a nickel resin. The produced protein sequence is presented in the FIG. 1B.

### Anti-VEGF_{222/NF} Antibody Production

Anti-VEGF_{222/NF} polyclonal antibodies were produced in Rabbit by ProteoGenix (Schiltigheim, France). Two peptides were used for rabbit immunization and their sequences are presented in FIG. 2. Briefly, peptides-coding sequences were conjugated to KLH-coding one. Rabbit were immunized for 51 days and antibodies production was determined by ELISA assay.

### Plasmids

To generate lentiviral VEGF₁₆₅ or VEGF_{222/NF} expression plasmids, a gene synthesis of VEGF₁₆₅ or VEGF_{222/NF} was performed (Eurofins Genomics) and subcloned into pLenti6.3/TO/V5-GW/LacZ-Blasti (ThermoFischer) via SpeI and XhoI restriction sites, replacing the LacZ gene. For these constructs, the C-terminal V5-tag was not in frame. To generate lentiviral shRNA plasmids, pLKO.1-TRC cloning vector, a kind gift from David Roots, has been used. pLKO.1-TRC contains a 1.9 kb stuffer released by Agel and EcoRI digestion. shRNA oligos were annealed and ligated into the Agel and EcoRI sites in place of the stuffer. The following shRNA sequences were used: shScramble: 5′-CCTAAGGITAAGTCGCCCTCG-3′ (SEQ ID NO: 26); shVEGF #1: 5′-GCGCAAGAAATCCCGGTATAA-3′ (SEQ ID NO: 27) shVEGF #2: 5′-AGGGCAGAATCATCACGAAGT-3′ (SEQ ID NO: 28); shVEGF_{NF} #1: 5′-GCCTTTGTITTCCATTTCC-3′ (SEQ ID NO: 29); shVEGF_{NF} #2: 5′-CATITCCCTCAGATGTGACAA-3′(SEQ ID NO: 30); shNRP1: 5′-TGTGGATGACATTAGTATTAA-3′(SE ID NO: 31); shNRP2: 5′-CCTCAACITCAACCCTCACIT-3 (SEQ ID NO: 32)′. All plasmids were verified by Sanger sequencing (Eurofins Genomics).

### Lentiviral Production and Transduction

Lentivirus were produced by triple transfection of HEK-293T cells with a lentiviral transfer vector (pLenti6.3 for overexpression experiments and pLKO.1 for shRNA experiments), and the packaging plasmids psPAX2 and pMD2.G at a 0.3:0.3:0.1 ratio. Transfection was performed using JetPEI® reagent as recommended by the manufacturer Polyplus transfection. The viral supernatant was collected 48 hours following transfection, filtered through a 0.22 μm filter, and added to target cells.

### Cell Proliferation Assays

For endothelial cell proliferation assays, TIME or HDLECs cells (50.000 and 25.000, respectively) were seeded in 6-well plates in Endothelial Cell Growth Medium (Promocell) containing 0.5% FBS. Twenty-four hours later, cells were treated with VEGF₁₆₅ (100 ng/ml) or VEGF_{222/NF} (100 ng/ml) (Day 0) and were counted at day 0, 1, 3, 5 and 7. Results were expressed as fold increase considering day 0 as the reference. For ACHN proliferation, ACHN-LacZ, ACHN-VEGF₁₆₅, ACHN-VEGF_{222/NF} cells were seeded (20.000) in 6-well plates in DMEM medium containing 0.5% FBS. Cells were counted at day 0, 1, 3, 5 and 7. Results are expressed as fold increase considering day 0 as the reference.

### Clonogenicity

For evaluation of colony-forming capability, colony formation assays were performed. ACHN and 786-O cells were washed twice with PBS, and reseeded at a density of 8.000 (ACHN) or 4.000 (786-0) cells/well in 6-wells plates. Twenty-four hours later, cells were transduced with two different sequences targeting VEGF, shVEGF (#1 and #2) or shVEGF_{222/NF} (#1 and #2). Twenty-four hours after, media were changed. After 7 days, colonies were stained with 0.1% crystal violet. The plates were photographed.

### Migration Assay

TIME cells were cultured to confluency in 6-well plates. Cells were serum-starved for two hours and the cell monolayer was disrupted to produce a scratch-wound using a sterile disposable plastic pipette tip of 10 mm diameter and rinsed with PBS before treatment with VEGF₁₆₅ (100 ng/ml) or VEGF_{222/NF} (100 ng/ml). Images were captured immediately after scratching (0 hr) using phase contrast microscopy (Evos™ xl core, ThermoFischer), and at 3, 6, 9 and 12 hours. Images were analyzed using Java-based ImageJ34 and the distance measured at 0, 3, 6, 9 and 12 hours.

### Endothelial Permeability Assays

In vitro permeability assessed were performed as previously described (Gasmi et al., 2002, Complete structure of an increasing capillary permeability protein (ICPP) purified from Vipera lebetina venom. ICPP is angiogenic via vascular endothelial growth factor receptor signalling. J Biol Chem 277, 29992-29998). Briefly, TIME cells were grown in vascular cell basal medium (ATCC® PCS-100-030™) supplemented with microvascular endothelial cell growth kit (ATCC® PCS-100-041™) on the membrane of 6.5 mm transwell insert (0.4 μm pore size, CORNING®). Once a monolayer is formed, cells were serum-starved for two hours and cells were then treated with VEGF₁₆₅ (100 ng/mL) or VEGF_{222/NF} (100 ng/ml) in the upper chamber for twenty minutes. The medium was then aspirated and refilled with streptavidin-HRP containing medium (1/200, R&D systems) for five minutes. The inserts were removed and 20 μl of media from the lower chamber was transferred to a 96 well-plate. TMB substrate (50 μL, (Sigma Aldrich)) was added for five minutes and the reaction was stopped according to the manufacturer's protocol (BIOLEGEND®). Absorbance was acquired at 450 nm with an ELISA reader (ThermoScientific, Multiskan FC). Relative permeability was expressed as percent of control.

### Immunoblotting

Cells were lyzed in Laemmli buffer containing 2% SDS, 10% Glycerol, 60 mM Tris-HCl (pH 6.8), 1× Halt™ phosphatase inhibitor cocktail (Thermo Fischer). DNA was fragmented by sonication. Lyzates were mixed to a 0.002% bromophenol blue and 100 mM DTT solution and then heated to 96° C., separated by SDS-PAGE, and transferred to PVDF membrane (Millipore). Membranes were blocked in 5% milk in PBS, probed with the indicated antibodies, and reactive bands visualized using chemiluminescent Western Immobilon™ HRP substrate (MERCK MILLIPORE®).

### RT-PCR and RT-qPCR

RT-PCR and RT-qPCR analyses were carried out on human tissue mRNA (BIOCHAIN®) and on cancer cell lines. mRNAs were prepared with a Nucleospin RNA kit (Macherey-Nagel), and cDNA synthesis was performed with a Maxima First Strand CDNA Synthesis Kit for RT-qPCR, with dsDNase (Fischer scientific), according to the manufacturers' instructions.

PCR analysis was performed on Biometra thermal cycler with PrimeSTAR GXL DNA polymerase (Takara Bio). Quantitative PCR analysis was performed on Applied Biosystems StepOnePlus™ System with TB Green® premix Ex Taq™ (Tli RNase H Plus) (Takara Bio) reagents. All samples were assayed in triplicate. The primers used are listed in Table S1. Relative expression levels were determined with the dCt method and normalized to the 36B4 reference gene.

TABLE S1
Primers used for PCR and qPCR

| | | SEQ | | SEQ |
|---|---|---|---|---|
| | | ID | | ID |
| | | NO | | NO |
| qPCR | Forward | | Reverse | |
| VEGF222/NF | GCCTTTGTTTTCCATTTCCCT | 33 | TCTGTCGATGGTGATGGTGTG | 34 |
| (hVEGF_{xxx/} | | | | |
| _{NFTotal}) | | | | |
| hVEGF | TTTCTGCTGTCTTGGGTGCATTG | 35 | ACCACTTCGTGATGATTCTGC | 36 |
| total | G | | CCT | |
| hVEGF₁₂₁ | ATCTTCAAGCCATCCTGTGTGC | 37 | TGCGCTTGTCACATTTTTCTTG | 38 |
| human | CAGATTGGCTACCCAACTGTT | 39 | GGCCAGGACTCGTTTGTACC | 40 |
| 36b4 | | | | |
| mouse | AGATTCGGGATATGCTGTTGG | 41 | TCGGGTCCTAGACCAGTGTT | 42 |
| 36b4 | C | | C | |
| mCD31 | ACGCTGGTGCTCTATGCAAG | 43 | TCAGTTGCTGCCCATTCATCA | 44 |
| mVEGFR2 | TTTGGCAAATACAACCCTTCA | 45 | GCAGAAGATACTGTCACCA | 46 |
| | GA | | CC | |
| maSMA | GTCCCAGACATCAGGGAGTA | 47 | TCGGATACTTCAGCGTCAGG | 48 |
| | A | | A | |
| CD45 | CCTCACCTGCTCCTCAAACTT | 49 | CATCCACTTTGCCCTCTGCTT | 50 |
| | C | | C | |
| IL-1 | GCAACTGTTCCTGAACTCAAC | 51 | ATCTTTTGGGGTCCGTCAACT | 52 |
| | T | | | |
| TNFα | CTATGTCAGCCTCTTCTC | 53 | CATTTGGGAACTTCTCATCC | 54 |
| iNOS | TCACCTTCGAGGGCAGCCGA | 55 | TCCGTGGCAAAGCGAGCCA | 56 |
| | | | G | |
| TGFβ | ATACGCCTGAGTGGCTGTCT | 57 | CGCTGAATCGAAAGCCCTGT | 58 |
| ARG1 | GATTATCGGAGCGCCTTTCT | 59 | CCACACTGACTCTTCCATTCTT | 60 |
| PD-1 | ACCCTGGTCATTCACTTGGG | 61 | CATTTGCTCCCTCTGACACTG | 62 |
| TIM-3 | TCAGGTCTTACCCTCAACTGTG | 63 | GGCATTCTTACCAACCTCAA | 64 |
| | | | ACA | |
| CTLA-4 | TTTTGTAGCCCTGCTCACTCT | 65 | CTGAAGGTTGGGTCACCTGT | 66 |
| | | | A | |
| VEGF_{NFtotal} | CCTTTGTTTTCCATTTCCCT | 70 | TCTGTCGATGGTGATGGTGTG | 71 |
| hVEGF₁₆₅ | TGTTTGTACAAGATCCGCAGA | 74 | CTCGGCTTGTCACATCTGCA | 75 |
| | CGTG | | AGTACG | |
| hVEGF_{168/NF} | CCCACTGAGGAGTCCAACAT | 76 | GGAAAACAAAGGCTGCATT | 77 |
| | CAC | | CACATTTG | |
| hVEGF_{178/NF} | GCGGATCAAACCTCACCAAG | 78 | GGAAAACAAAGGTTTTCTTGT | 79 |
| | G | | CTTGCTC | |
| hVEGF_{202/NF} | GCGGATCAAACCTCACCAAG | 80 | GGAAAACAAAGGACGCTC | 81 |
| | G | | CAGGACTTATAC | |
| hVEGF_{222/NF} | CAAGACAAGAAAATCCCTGT | 82 | GGAAATGGAAAACAAAGGC | 83 |
| | GGGC | | TGCAAGTAC | |
| hVEGF_{240/NF} | CAAGAAATCCCGTCCCTGTG | 84 | GGAAATGGAAAACAAAGGC | 85 |
| | GG | | TGCAAGTAC | |
| hVEGF_{246/NF} | CCTGGAGCGTTCCCTGTGG | 86 | GGAAATGGAAAACAAAGGC | 87 |
| | | | TGCAAGTAC | |
| hVEGF_{263/NF} | CCTGGAGCGTGTACGTTGGTG | 88 | GGAAATGGAAAACAAAGGC | 89 |
| | | | TGCAAGTAC | |
| mVEGFR3 | CGAGTCGGAGCCTTCTGAGG | 90 | GCAGTCCAGCAATAGGGG | 91 |
| | | | GT | |
| mLYVE-1 | CAG | 92 | CGCCCATGATTCTGCATGTA | 93 |
| | CACACTAGCCTGGTGTTA | | GA | |
| mPROX1 | TGCGTGTTGCACCACAGAATA | 94 | AGAAGGGTTGACATTGGAGT | 95 |
| | | | GA | |
| PCR | Forward | | Reverse | |
| VEGF_{222/NF} | GCCTTTGTTTTCCATTTCCCT | 33 | TCTGTCGATGGTGATGGTGTG | 34 |
| VEGF | TTTCTGCTGTCTTGGGTGCATTG | 35 | ACCACTTCGTGATGATTCTGC | 36 |
| | G | | CCT | |
| 36b4 | GGCGACCTGGAAGTCCAAC | 67 | CCATCAGCACCACAGCCTT | 68 |
| | | | C | |
| VEGF_{NFtotal} | GCGGATCAAACCTCACCAAG | 72 | GCTTGTCACATCTGAGGGAA | 73 |
| | G | | ATG | |

### Immunofluorescence

Tumor sections (5-μm cryostat sections) were fixed in 4% paraformaldehyde for twenty minutes at room temperature and blocked in 1% donkey serum in Tris-buffered saline (TBS) for two hours. Sections were then incubated overnight with anti-rabbit LYVE-1 polyclonal (Ab14917, 1:200; Abcam) or rat monoclonal anti-mouse CD31 (clone MEC 13.3, 1:1000; BD Pharmingen) and monoclonal anti-mouse a-smooth muscle actin (aSMA A2547, 1:1000; Sigma) antibodies. Preparations were mounted and analyzed with a Leica microscope (Leica DMI4000B) and counted at a 10× magnification. Results are expressed as the number of vessels per mm² of sections.

### ELISA Assay

RCC cell lines were seeded (500.000) in 6-well plates and grown in 0.5% FBS containing DMEM medium for 48 h. The production of VEGF₁₆₅ and VEGF_{222/NF} was assessed by ELISA. VEGF₁₆₅ assay was carried out with the human VEGF₁₆₅ standard TMB ELISA development kit (PEPROTECH®) according the to manufacturer recommendations. For VEGF_{222/NF}, the anti-VEGF_{222/NF} antibody (clone #2, at 1.5 μg/mL) was coated in PBS overnight at 4° C. Saturation was obtained with a PBS-2% BSA solution for one hour at room temperature. Samples were incubated in PBS-0.5% BSA-0.05% Tween for one hour at room temperature. The detection antibody from the VEGF₁₆₅ standard TMB ELISA development kit (PEPROTECH®) was used and revelation was assessed as for the VEGF₁₆₅. Results are expressed as picograms of VEGF_{222/NF} per million of cells per 48 h. To determine bevacizumab affinity for VEGF and VEGF_{222/NF}, saturation binding was determined on 96-well plates coated with human VEGF₁₆₅ and VEGF_{222/NF} recombinant protein (100 ng/well). Serial dilutions of bevacizumab were incubated for 1 h at room temperature before being washed 5 times with PBS-Tween 0.01%. Bevacizumab binding was determined with an anti-human HRP-conjugate antibody (goat anti human IgG, Thermo Fischer scientific). Bevacizumab binding curves were fitted using a nonlinear regression equation (specific binding: y=Bmax×x/(KD+x), with x being the bevacizumab concentration, KD being the dissociation constant, and Bmax being the maximum number of binding sites, or receptor density) (GraphPad Prism, version 8, software), to determine KD values. Bevacizumab binding was normalized to Bmax for graphic representation.

### In Vivo Experiments

### In Vivo Permeability Assay

6-weeks old female BALB/cJRj mice (n=15) were injected with 1% Evans blue dye (100 μL) in the tail vein. Then, PBS, VEGF₁₆₅ (500 ng/ml) or VEGF_{222/NF} (500 ng/ml), were intradermally injected into the right and the left ears (10 μL) using a 30-gauge needle. Twenty minutes after, the animals were euthanized, and the dye was extracted from the ears in formamide at 56° C. for 48 h. The intensity of the reaction was quantified by reading the samples at a wavelength of 620 nm. Ears were dried with 100% ethanol and were weighted. Results are expressed in nanograms of dye per milligram of dry tissue.

### In Vivo Plug Assay

6-weeks old female NMRI mice (n=15) were used for this experiment. PBS, VEGF₁₆₅ (200 ng), VEGF_{222/NF} (200 ng) were embedded in MATRIGEL® growth factors reduced and injected subcutaneously in the right and left flank of the mice (2 MATRIGEL® plugs per mice) in a 200 μL volume. MATRIGEL® plugs were recovered after two weeks for hemoglobin quantity analysis. Plugs hemoglobin content was assessed with the hemoglobin assay kit (Sigma Aldrich) and experiments were conducted according to the manufacturer recommendations. Results are expressed in micrograms of hemoglobin per milligram of MATRIGEL®.

### Zebrafish Studies

Zebrafish embryos were dechorionated with help of a sharp tip forceps and anesthetized with 0.04 mg/ml of tricaine (MS-222. Sigma). Anesthetized embryos were transferred onto a modified agarose gel for microinjection. Before injection, tumor cells were labelled with 2 mg/ml of 1,1-Dioctadecyl-3.3.3.3-tetramethylindocarbocyanine perchlorate (Dil, Fluka, Germany). Approximately 100-500 tumor cells were resuspended in serum-free DMEM (Gibco) and 5 nL of tumor cell solution were injected into the perivitelline cavity of each embryo using an Eppendorf microinjector (FemtoJet 5247. Eppendorf and Manipulator MM33-Right, Maizhauser Wetziar). Non-filamentous borosilicate glass capillaries needles were used for the microinjection (1.0 mm in diameter, World Precision Instruments, Inc.). After injection, the fish embryos were immediately transferred into housing-keeping water. Injected embryos were kept at 28° C. and were examined at 24, 48 and 72 h for monitoring metastasis using a fluorescent microscope (Nikon Eclipse C1).

### Tumor Experiments in Immunocompromised Mice

For immunocompromised mice, two millions of ACHN-LacZ (n=10). ACHN-VEGF₁₆₅ (n=10), ACHN-VEGF_{222/NF} (n=10) were prepared in a 1:1 PBS/Matrigel Growth factor reduced (CORNING®). Tumor cells (200 μL) were injected subcutaneously into the right and the left flank of 5-week-old NMRI female mice (Janvier Labs). The tumor volume was monitored once a week for ten weeks and was determined with a caliper (volume=L*12*0.5). At the end of the experiment, mice were sacrificed, and tumor were photographed using Zeiss AXIO Zoom.V16 microscope with a×4 magnification. Afferent tumor blood vessel diameter was measured using Image J software and analyzed at the first, second and third quartile.

### Patient Analyzes

Informed consent was obtained from all individual participants included in the study. All patients gave written consent for the use of tumor and blood samples for research. This study was conducted in accordance with the Declaration of Helsinki. Primary tumor samples of 93 non-metastatic (M0) ccRCC patients were obtained from the Rennes and Bordeaux University Hospitals (UroCCR group, NCT03293563). For metastatic patients, the population of the study included 47 ccRCC patients from the prospective SUVEGIL (NCT00943839) and TORAVA (NCT00619268) trials and from a retrospective cohort from Pavia (Italy). Metastatic patients received oral sunitinib (50 mg per day) once a day for 4 weeks (on days 1 to 28), followed by 2 weeks of treatment interruption. Sunitinib was continued in the absence of disease progression or unacceptable toxicity. Blood samples were collected before the beginning of the treatment (TO). Blood samples were centrifuged (10 000 g for 10 min) and the plasmas were collected and conserved at −80° C. Plasmatic levels of VEGF and VEGF_{222/NF} were determined by ELISA as described in the ELISA method details section. PFS and OS were calculated from patient subgroups with VEGFA or VEGF_{222/NF} plasmatic levels that were less or greater than the third quartile value.

### Statistical Analysis

Statistical analysis was carried out using GraphPad Prism 8. Data were expressed as mean±SEM and were compared using an unpaired Mann-Whitney test for intergroup analysis. For patients: The Student's t-test was used to compare continuous variables and chi-square test, or Fisher's exact test (when the conditions for use of the χ2-test were not fulfilled), were used for categorical variables. DFS was defined as the time from surgery to the appearance of metastasis. PFS was defined as the time between surgery and progression, or death from any cause, censoring live patients and progression free at the last follow-up. OS was defined as the time between surgery and the date of death from any cause, censoring those alive at the last follow-up. The Kaplan-Meier method was used to produce survival curves and analyses of censored data were performed using Cox models. Significance was defined as P<0.05.

### Results

### Example 1: Identification and Expression Profile of a Novel VEGF Splice Variant

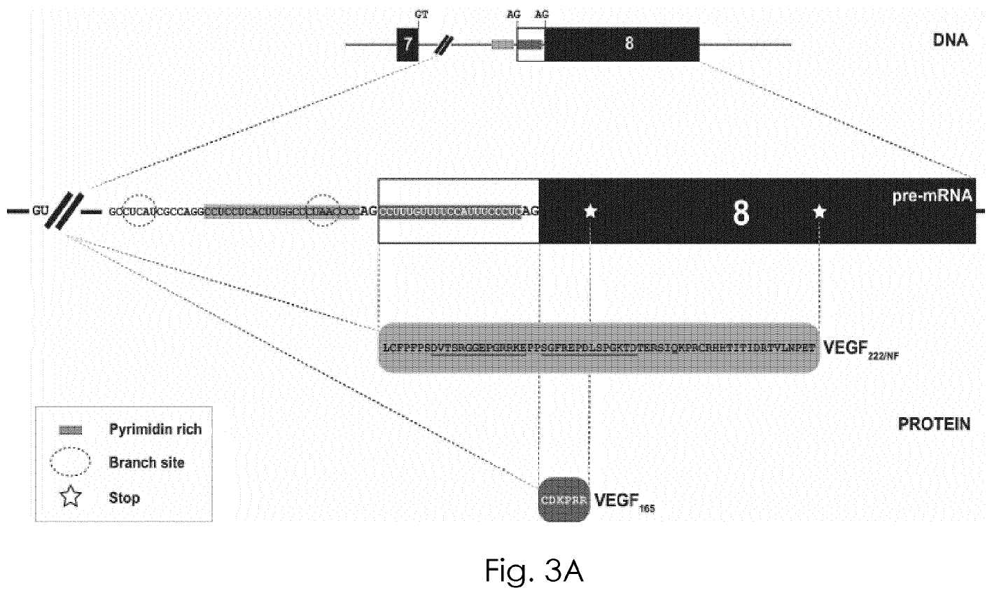

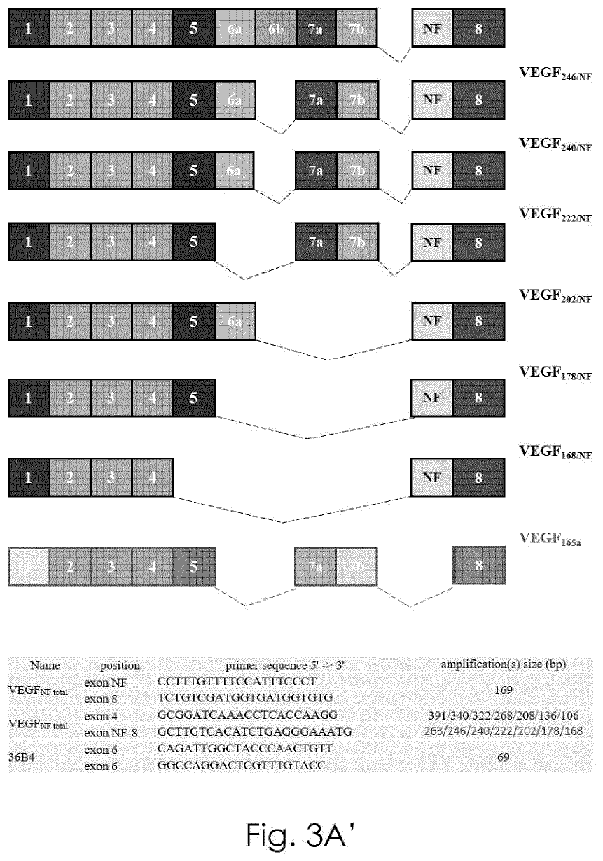

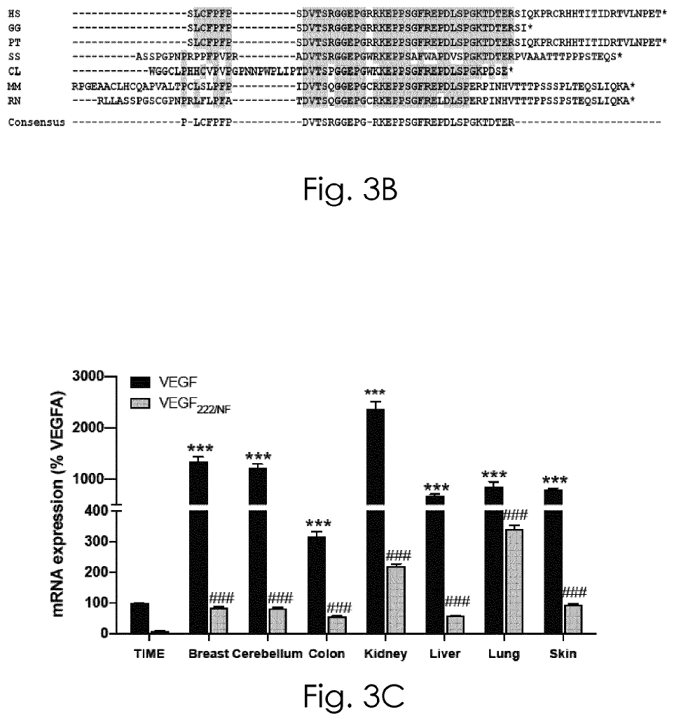

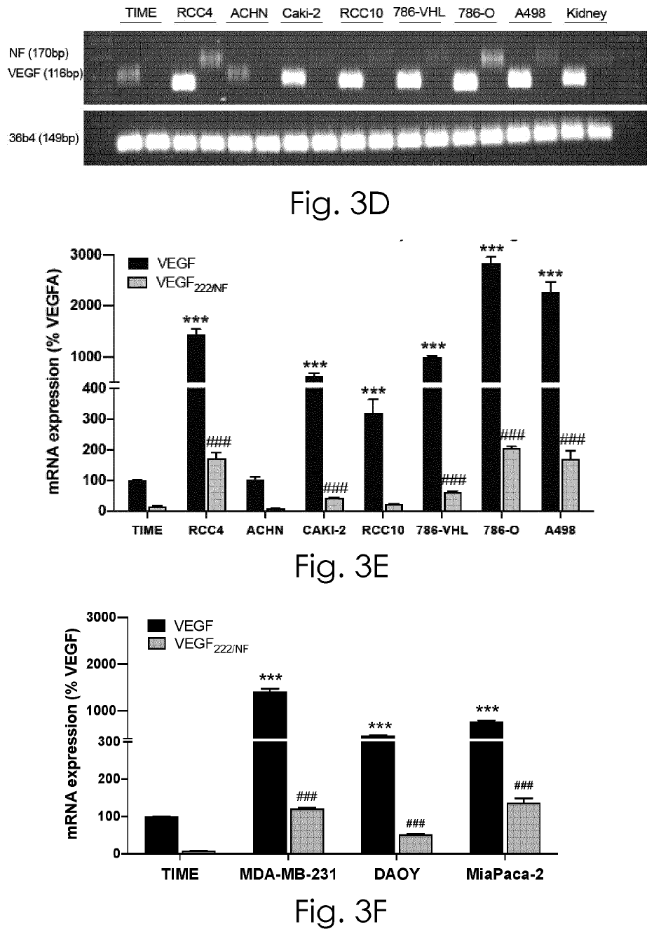

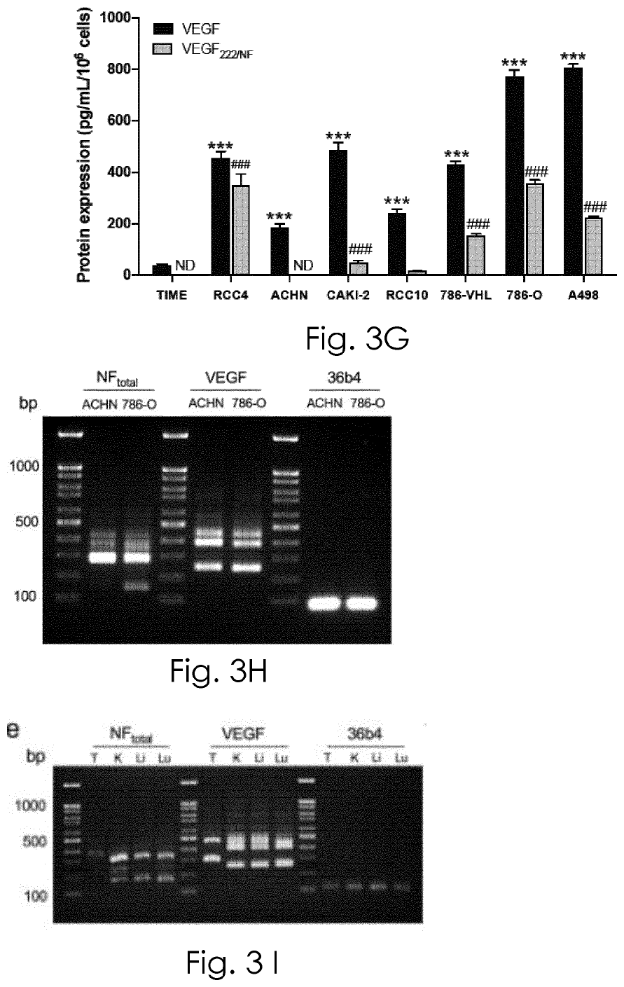

Bio-informatic analysis of the human VEGF gene sequence revealed the existence of a consensus splice acceptor site located 21 bp upstream the conventional AG splice acceptor site in the seventh intron. The presence of a consensus pyrimidine tract (in light grey) and of a consensus branch site is consistent with the presence of an alternative and functional splice acceptor (FIG. 3A). The insertion of these 23 bp (including AG), creates a new open reading frame allowing the translation to occur in the domain considered as the 3′ untranslated region (3′UTR) of the VEGF mRNA. The mRNA resulting from this alternative splicing, codes for a new VEGF isoform of 248 amino acids from the initiation methionine. The mRNAs resulting from this alternative splicing, code for seven new VEGF isoforms (FIG. 3A′). According to the international nomenclature, removal of the signal peptide gives rise to the VEGF₂₂₂ New Form, VEGF_{222/NF}: VEGF_{168/NF}, VEGF_{178/NF}, VEGF_{202/NF}, VEGF_{222/NF}. VEGF_{240/NF}. VEGF_{246/NF} and VEGF_{263/NF}.

An in-depth analysis of this domain of intron 7 and the beginning of exon 8 was performed using Genomnis bio-informatic platform (https://hsf.genomnis.com, Online Resource 3B). The Human Splicing Finder system identified all splicing elements including both acceptor and donor splice sites, branch points and auxiliary splicing signals (ESE and ESS). This analysis highlighted two strong branching points gcctcat (value=94,24) and tcctcac (value=98,24) upstream NF motif. The NF exon splicing acceptor site (CV value of 78,1) is a less efficient site as compared to the exon 8 acceptor site (value 85,83). The presence of multiple regulatory elements (ESS, ESE, splice acceptor site) revealed the complexity of the splicing mechanisms in this key region and re-enforces the hypothesis of the existence of alternative VEGF isoforms depending on the NF acceptor site. The alternative acceptor splice is located at different distances from the conventional AG in different species. However, the frameshift in the reading frame allows the translation in the region corresponding to the 3′UTR. Parts of the resulting amino acid sequence is highly conserved between several species but differs in its length (FIG. 3B).

The C-terminal fragments of VEGF_{222/NF} of the different specifies correspond to the following sequences disclosed in FIG. 3B and numbered SEQ ID NO: in the following table:

TABLE S2
C-terminal fragments of VEGF222/NF of the different specifies

| Sequence | SEQ ID NO: |
|---|---|
| C-terminal Fragment 1 of VEGF222/NF of | 13 |
| Homo Sapiens (HS), Gorilla Gorilla (GG) | |
| and Pan Troglodytes (PT) | |
| C-terminal Fragment 2 of VEGF222/NF of | 14 |
| Homo Sapiens (HS) and Pan Troglodytes | |
| (PT) | |
| C-terminal Fragment 2 of VEGF222/NF of | 15 |
| Gorilla Gorilla (GG) | |
| C-terminal Fragment 1 of VEGF222/NF of | 16 |
| Sus Scrofa (SS) | |
| C-terminal Fragment 2 of VEGF222/NF of | 17 |
| Sus Scrofa (SS) | |
| C-terminal Fragment of VEGF222/NF of | 18 |
| Canis Lupus (CL) | |
| C-terminal Fragment 1 of VEGF222/NF of | 19 |
| Mus Musculus (MM) | |
| C-terminal Fragment 2 of VEGF222/NF of | 20 |
| Mus Musculus (MM) | |
| C-terminal Fragment 1 of VEGF222/NF of | 21 |
| Rattus Norvegicus (RN) | |
| C-terminal Fragment 2 of VEGF222/NF of | 22 |
| Rattus Norvegicus (RN) | |
| C-terminal Fragment 1 consensus of | 23 |
| VEGF222/NF between species | |
| C-terminal Fragment 2 consensus of | 24 |
| VEGF222/NF between species | |
| C-terminal Fragment 3 consensus of | 25 |
| VEGF222/NF between species | |

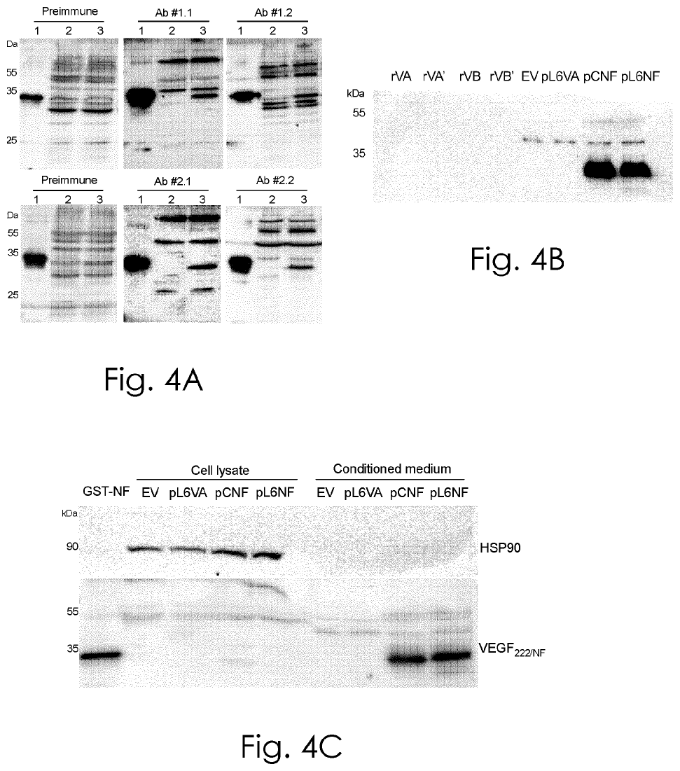

By designing specific primers, VEGF_{222/NF} mRNA was evidenced in all the evaluated human tissues with highest levels in kidney and lung (FIG. 3C). Its relative expression did not systematically coincide with those of VEGF (FIG. 3C). VEGF_{222/NF} mRNA expression was further detected in several cancer cell lines including ccRCC (FIGS. 3D, E), and breast, pancreatic carcinoma and medulloblastoma cell lines (FIG. 3F). Total VEGF_{XXX/NF} MRNA were amplified by RT-qPCR from human tissues (FIG. 3C) and RCC cell lines (FIG. 3E). Highest levels of VEGF_{XXX/NF} were found in kidney and lungs. VEGF_{XXX/NF} expression was found in several RCC cell lines (FIG. 3E). Importantly, part of the VEGF amplicon analyzed by classical (FIG. 3D) or qPCR, includes VEGF_{222/NF}. TWO Anti-VEGF_{222/NF} polyclonal antibodies were then produced against two conserved epitopes (FIG. 2) and fully characterized for their specificity (FIGS. 4A-C). These antibodies recognized a 30 kDa protein corresponding to the full-length VEGF_{222/NF}. VEGF_{222/NF} was detected in nearly all ccRCC cell lines except ACHN, with the highest levels in 786-0 and A498 cells. This expression pattern did not exactly follow those of VEGF (FIG. 3G). As for PCR experiment, the anti-VEGF antibodies used to detect total VEGF did not discriminate between conventional VEGFₓₓₓ and VEGF_{222/NF}.

These results unambiguously demonstrated the existence of a new VEGF splice variant encoding an unknown protein to date.

### Example 2: VEGF_{222/NF} Induces Endothelial Cell Proliferation, Migration and Promotes Vascular Permeability and Angiogenesis

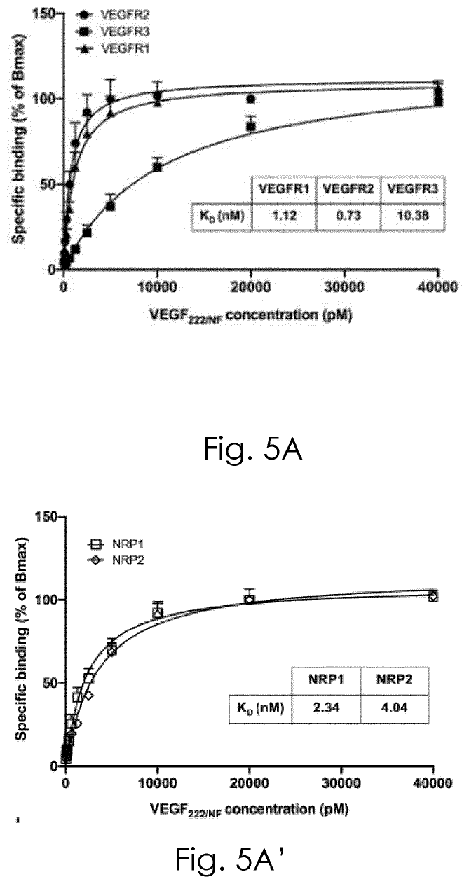

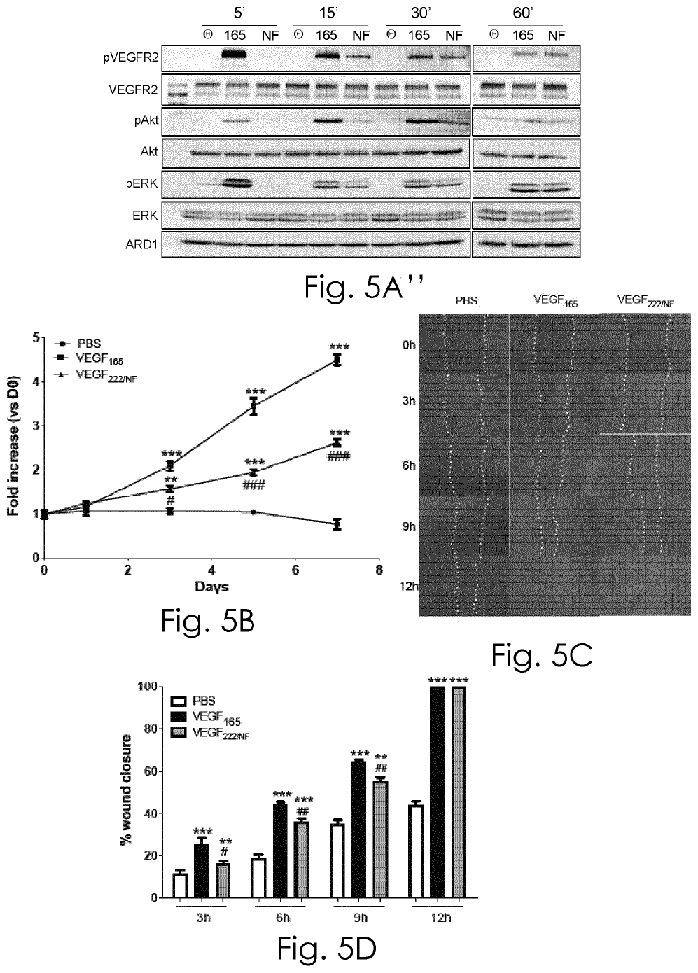

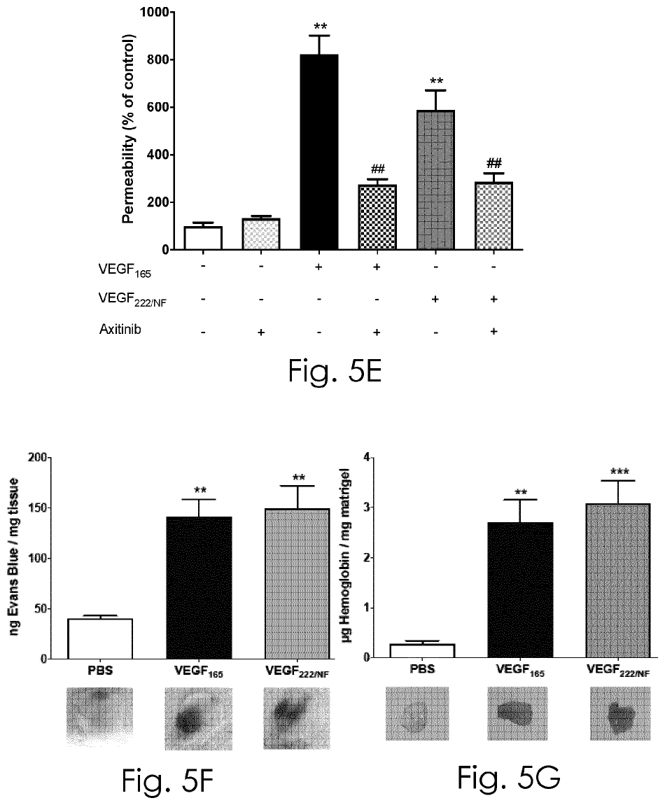

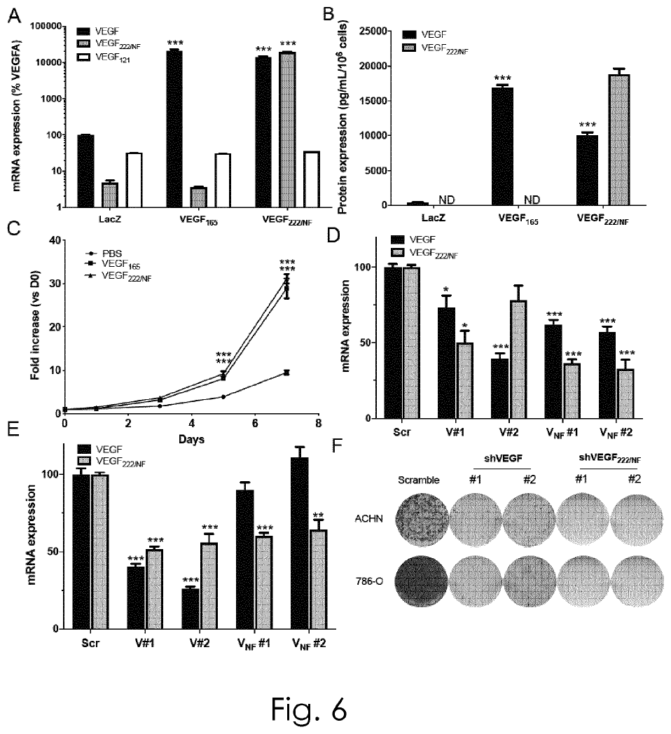

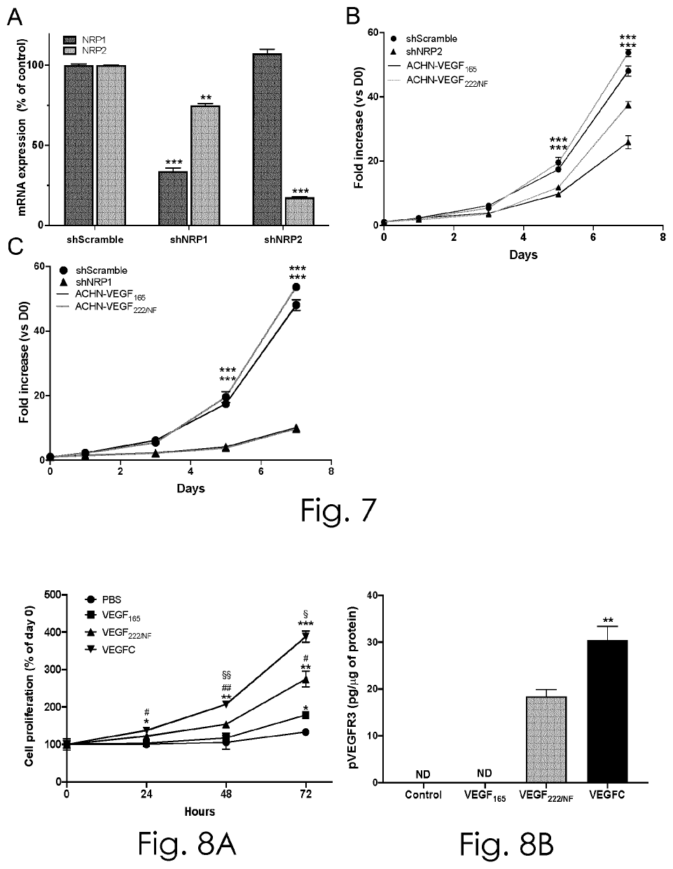

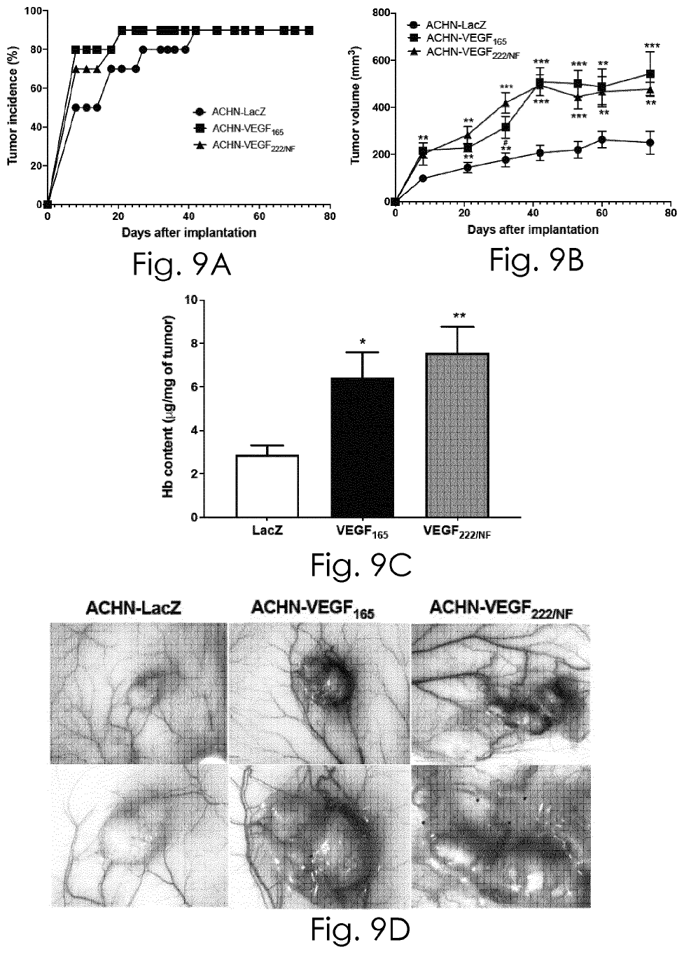

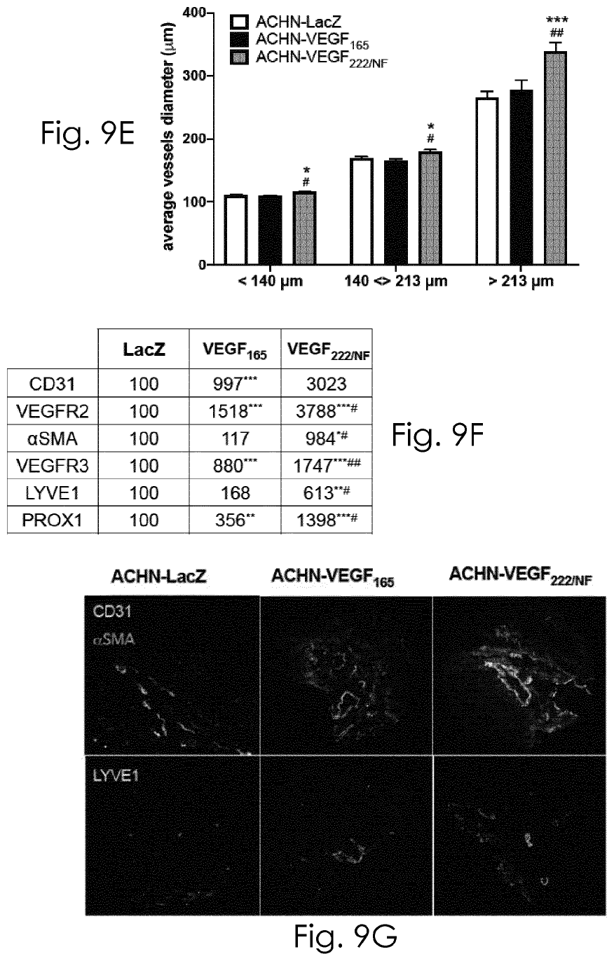

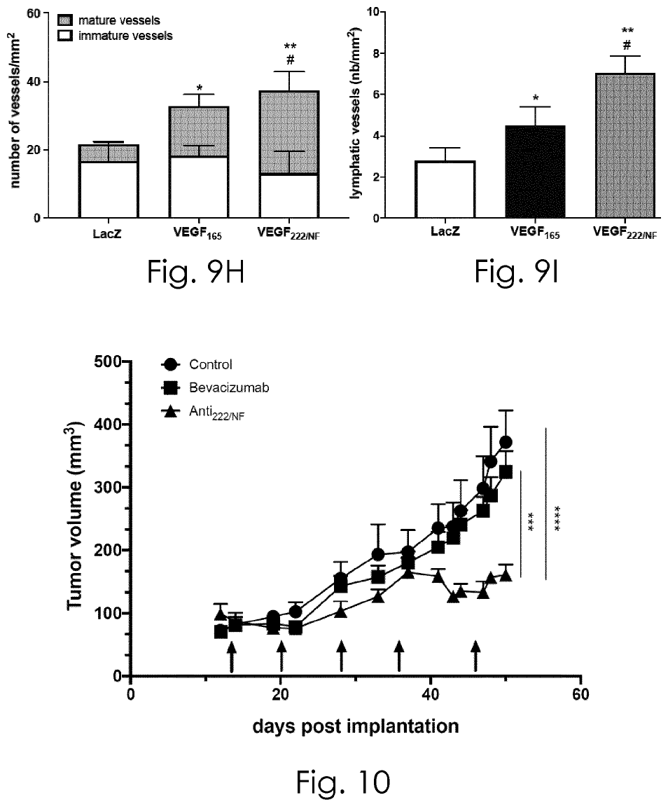

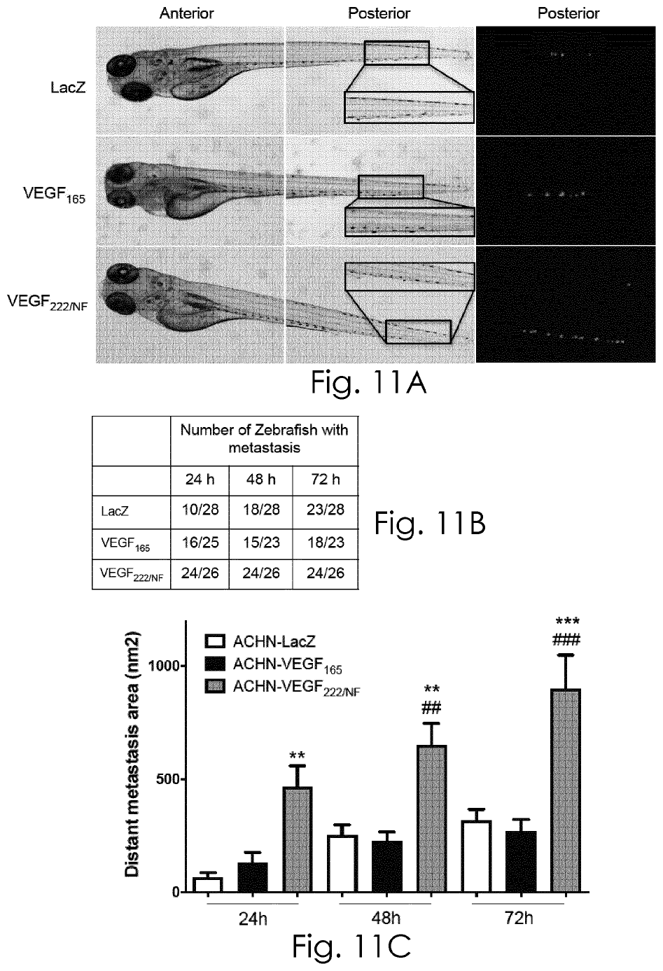

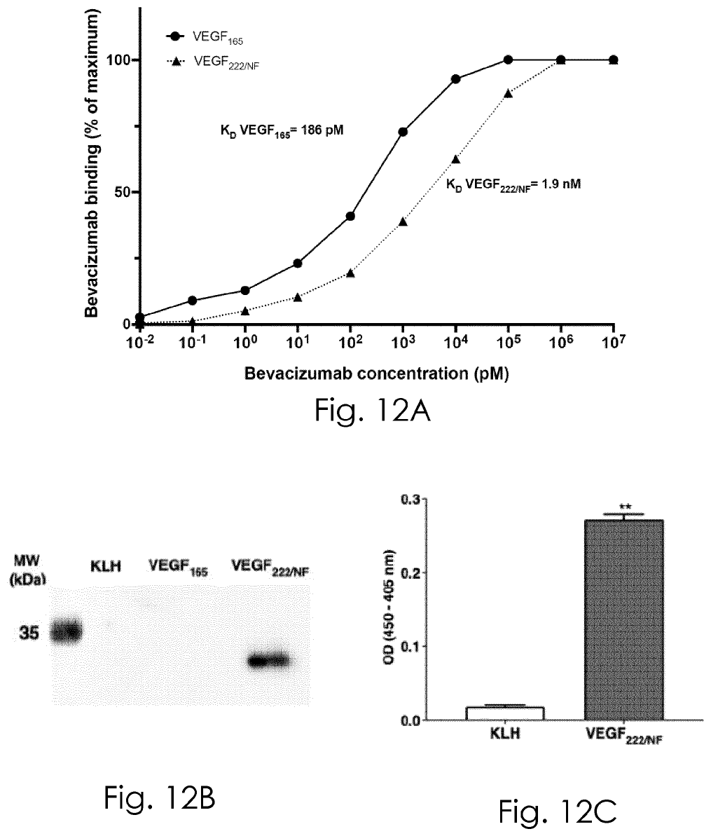

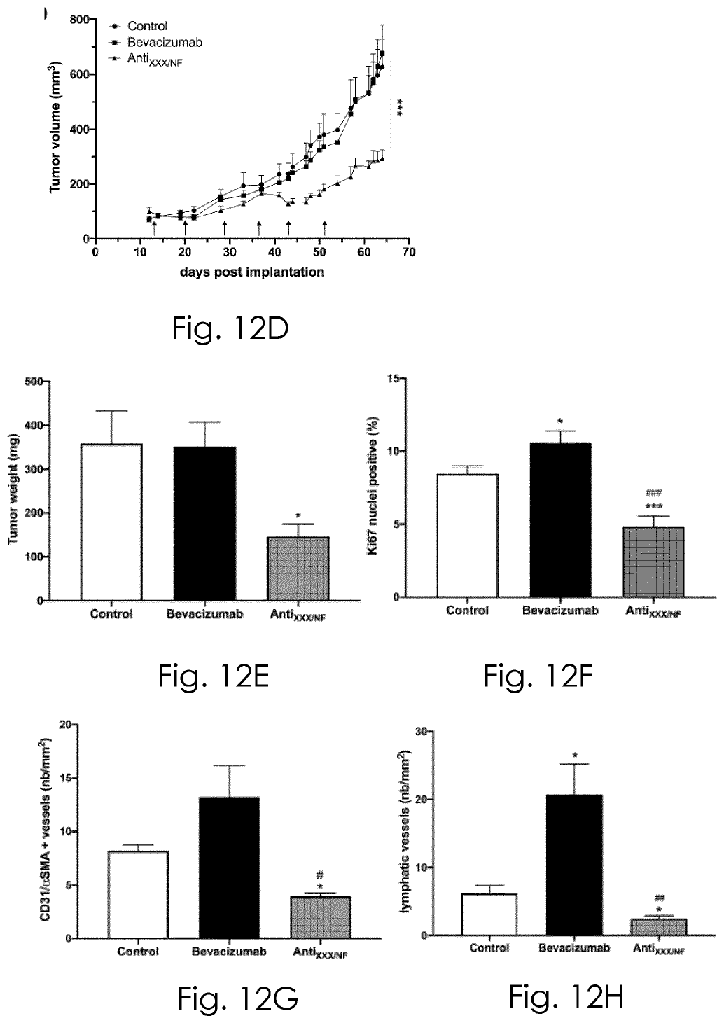

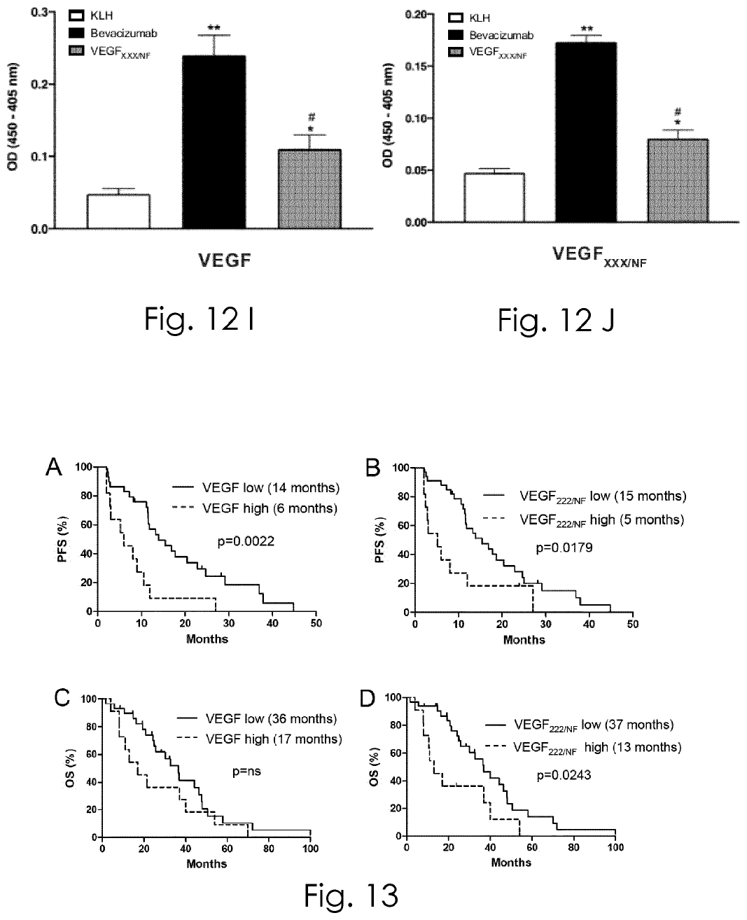

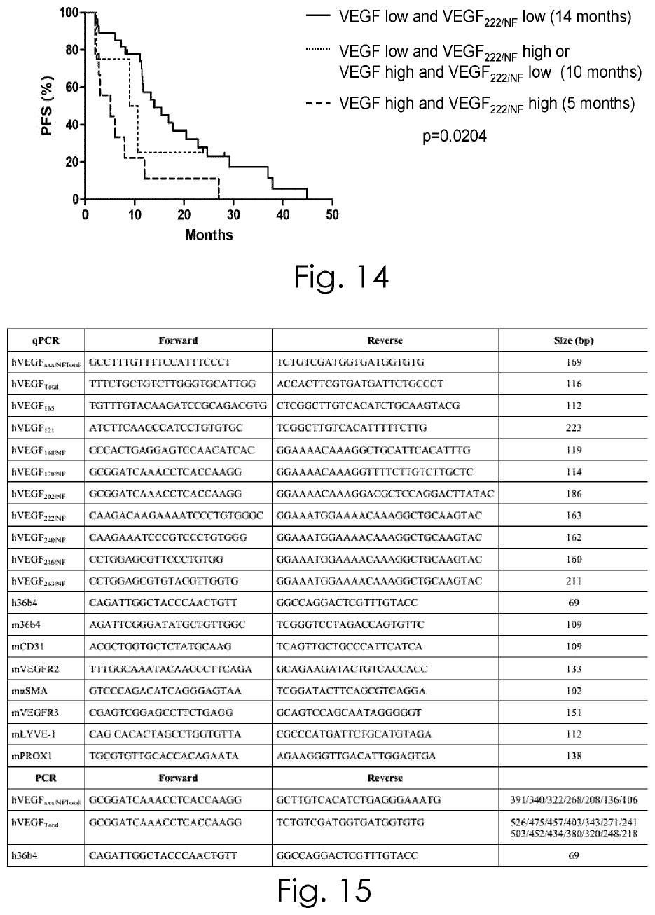

The ability of VEGF_{222/NF} to specifically bind VEGFRs and co-receptors was first assessed by saturation binding experiments. VEGF_{222/NF} binds VEGFR1 and VEGFR2 with a nanomolar-range affinity, with a respective KD of 1.12 and 0.73 nM (FIG. 5A). VEGF_{222/NF} was also found to bind VEGF co-receptors neuropilin 1 and 2 (NRP1 and NRP2) with a same affinity-range (FIG. 5A′). Despite a lower affinity, VEGF_{222/NF} also binds VEGFR3 (K_{D}=10.38 nM). These results prompted us to investigate the effects of VEGF_{222/NF} on physiological angiogenesis.

The inventors first compared the effect of VEGF₁₆₅ and VEGF_{222/NF} on physiological angiogenesis. VEGF₁₆₅ and VEGF_{222/NF} induced a sustained phosphorylation/activation of VEGFR2 and a subsequent ERK and AKT activation in endothelial cells (ECs) with a small delay in the activation process for VEGF_{222/NF} (FIG. 5A″). VEGF_{222/NF} stimulated the proliferation of serum- and growth factors-starved ECs to a lesser extent as compared to VEGF₁₆₅ from day 3 (P<0.05) to day 7 (P<0.001, FIG. 5B). The migration of ECs is a critical step of angiogenesis. ECs migrated more slowly in response to VEGF_{222/NF} as compared to a stimulation by VEGF₁₆₅ in a wound healing assay (P<0.05 at 3 h, P<0.01 at 6 h and 9 h, FIGS. 5C,D). The pro-permeability property of VEGF_{222/NF} is less important as compared to VEGF₁₆₅ but it is equivalently inhibited by the VEGFR1/2/3 inhibitor, AXITINIB® (FIG. 5E). The positive effects of VEGF_{222/NF} on ECs permeability were confirmed in vivo by measuring the extravasation of Evans blue dye in mice ears (FIG. 5F). This pro-permeability activity was 3-fold more important as compared to control conditions (P<0.01) and similar to those of VEGF₁₆₅. Matrigel® plug assays showed that the reddish aspect and the hemoglobin content of VEGF₁₆₅ and VEGF_{222/NF} plugs were equivalent (FIG. 5G).

These experiments further demonstrated the pro-angiogenic properties of VEGF_{222/NF} that were equivalent to those of VEGF₁₆₅.

### Example 3: VEGF_{222/NF} Promotes the Survival and the Proliferation of ccRCC Cells

The role of VEGF in cancer is not limited to angiogenesis and vascular permeability. VEGF-mediated signalling occurs in tumor cells and this signalling contributes to key aspects of tumorigenesis. The proliferative effect of VEGF_{222/NF} was first evaluated in ccRCC ACHN cells that do not express VEGF_{222/NF}. The overexpression of VEGF_{222/NF} and VEGF₁₆₅ in ACHN cells was first confirmed by RT-qPCR analyzis and ELISA assays (FIGS. 6A,B). Overexpression of VEGF_{222/NF} or VEGF₁₆₅ did not affect those of another splice form of VEGF, VEGF₁₂₁ (FIG. 6A). VEGF₁₆₅ and VEGF_{222/NF} stimulated ACHN cell proliferation (P<0.001, FIG. 6C). The pro-proliferative effect of VEGF_{222/NF} was inhibited by decreasing the expression of the VEGFR2 co-receptor neuropilin 1 (NRP1) (FIGS. 7A-C). VEGF_{222/NF} stimulates an autocrine proliferation loop that involves, at least, the NRP1 pathway, which plays a key role in ccRCC cell proliferation (Cao et al., 2008, Neuropilin-1 upholds dedifferentiation and propagation phenotypes of renal cell carcinoma cells by activating Akt and sonic hedgehog axes. Cancer Res 68, 8667-8672). Two independent shRNA (shRNA #1 and #2) directed against VEGF or VEGF_{222/NF} (shRNA NF #1 and NF #2) downregulated the expression of their respective targets in ACHN (FIG. 6D) and 786-O cells (FIG. 6E). Downregulation of VEGF or VEGF_{222/NF} impaired cell proliferation and but only down-regulation of VEGF_{222/NF} induced cell death in clonogenicity assays (FIG. 6F).

These results strongly suggest that VEGF_{222/NF} is a key player of ccRCC cell survival and proliferation.

### Example 4: VEGF_{222/NF} Stimulates Human Dermal Lymphatic Endothelial Cells (HDLECs) Proliferation and Induces Phosphorylation of VEGFR3

The effect of VEGF_{222/NF} on lymphangiogenesis was first assessed by measuring its effect on the proliferation of human dermal lymphatic endothelial cells (HDLECs). In contrary to VEGF₁₆₅, VEGF_{222/NF} induced the proliferation of HDLECs (FIG. 8A). This effect can be explained by the VEGFR3 activation following VEGF_{222/NF} stimulation (FIG. 8B).

These results demonstrated the direct pro-lymphangiogenic effect of VEGF_{222/NF}.

To the inventors' knowledge, this is the first VEGF isoform that activates lymphangiogenesis through this receptor.

### Example 5: VEGF_{222/NF} Promotes Tumor Growth and Induces Optimal Tumor Angiogenesis, and Lymphangiogenesis

The comparison of the pro-tumoral activity of VEGF_{222/NF} and VEGF₁₆₅ was determined by generating experimental tumors with ACHN-VEGF_{222/NF} and ACHN-VEGF₁₆₅ cells in immunodeficient mice. A 100% incidence (percentage of mice with tumors) was reached at day 20 in ACHN-VEGF_{222/NF} and ACHN-VEGF₁₆₅ groups in comparison to 70% in the control group (FIG. 9A). Tumors generated with ACHN-VEGF_{222/NF} and ACHN-VEGF₁₆₅ were 2.5-fold bigger as compared to ACHN-LacZ control tumors (FIG. 9B). Tumor vascularization, assessed by testing the tumors' hemoglobin content, was 2.5-fold higher in ACHN-VEGF_{222/NF} and ACHN-VEGF₁₆₅ as compared to ACHN-LACZ tumors (P<0.01, FIG. 9C). A simple observation showed that the peri-tumoral vascularization and the vessel diameter were higher in the ACHN-VEGF_{222/NF} group in comparison to the ACHN-LacZ and ACHN-VEGF₁₆₅ groups (FIG. 9D, top). The quantification of blood vessels' diameter confirmed this observation; the average diameters of blood vessels reaching ACHN-VEGF_{222/NF} tumors, inferior to 140 μm, between 140 and 213 μm and superior to 213 μm (sizes corresponding to the first and third quartile and intermediate sizes between these two thresholds) were superior as compared to the diameters of vessels in the two other groups (FIG. 9E). Beside the blood vessel network, an unexpected dense lymphatic vessel network reached the ACHN-VEGF_{222/NF} tumors (black stars, FIG. 9D, bottom). Analysis of angiogenic and lymphangiogenic genes showed that VEGF_{222/NF} overexpression is associated with a more important increase in the levels of CD31, VEGFR2 and aSMA (angiogenesis) and VEGFR3, LYVE1 and PROX1 (lymphangiogenesis) in comparison to VEGF₁₆₅ overexpression (FIG. 9F). This observation suggests the induction of mature vessels covered by aSMA-positive pericytes and the development of a more important lymphatic network by the VEGF_{222/NF}. Immunofluorescence labelling with anti-CD31 and anti-aSMA confirmed a higher number of mature vessels (CD31+ and aSMA+) in VEGF_{222/NF} as compared to control and VEGF₁₆₅-expressing tumors (P<0.05, FIGS. 9G-H). Immunofluorescence labelling with LYVE1 also confirmed a denser lymphatic network in VEGF_{222/NF} tumors as compared to the two other groups (P<0.05, FIG. 91).

These results favored the notion that VEGF_{222/NF} is a more potent pro-angiogenic and a pro-lymphangiogenic factor as compared to VEGF₁₆₅.

### Example 6: VEGF_{222/NF} Inhibition Delays Tumor Growth of Experimental RCC

The anti-tumoral effect of VEGF_{222/NF} inhibition was assessed in experimental model of RCC. The treatment of 786-O-experimental tumors with anti-VEGF_{222/NF} antibodies delayed tumor growth from day 43 following implantation whereas bevacizumab did not (P<0.01, FIG. 10).

This result demonstrated the relevance of VEGF_{222/NF} inhibition specifically.

### Example 7: VEGF_{222/NF} Promotes Distant Metastasis in Zebrafishes

The zebrafish represents a unique experimental metastasis model for a dynamic study of cancer progression. Considering the role of VEGF_{222/NF} in tumor angiogenesis, the inventors next investigated the potential of VEGF_{222/NF}-expressing tumor cells to disseminate from the site of injection to the tails of zebrafishes. ACHN-LacZ, ACHN-VEGF_{222/NF} and ACHN-VEGF₁₆₅ were xeno-transplanted in the perivitelline zone and metastatic foci in the tail were determined at 24, 48 and 72 h. A significant enhanced dissemination at all the investigated time points was obtained for the ACHN-VEGF_{222/NF} group in comparison to the two others (FIGS. 11A,C). Moreover, an earlier dissemination was observed for the ACHN-VEGF_{222/NF} Cells (FIG. 11B).

These results suggest that the VEGF_{222/NF} more efficiently promotes metastatic dissemination.

### Example 8: Bevacizumab has a Lower Affinity for VEGF_{222/NF}

The presence of VEGF_{222/NF} is a plausible explanation of the reduced bevacizumab efficacy. For that purpose, the inventors designed a specific ELISA test to assess the affinity of bevacizumab to VEGF₁₆₅ and VEGF_{222/NF}.

This experiment showed that the affinity of bevacizumab for VEGF_{222/NF} is roughly ten-fold lower as compared to VEGF₁₆₅ (FIG. 12A).

Polyclonal antibodies directed against VEGF_{XXX/NF} were produced in mice and their specificity characterized (FIG. 12B and FIG. 12C). Anti-VEGF_{XXX/NF} antibodies significantly slowed-down the growth of experimental tumors generated with 786-O cells by 56% whereas the size of tumors in mice treated with bevacizumab was equivalent to those of the control group as already described (FIG. 12D). This result was consistent with a 60% decrease in the weight of tumors from mice treated with the anti-VEGF_{XXX/NF} antibodies (FIG. 12E). The number of proliferative Ki67-positive cells strongly decreased in these tumors but not in tumors from bevacizumab-treated mice (FIG. 12F). Besides the anti-proliferative effect, anti-VEGFₓₓₓ/NF decreased the intra-tumoral levels of CD31⁺/aSMA⁺ vessels (FIG. 12G). Moreover, anti-VEGF_{XXX/NF} decreased the number of lymphatic vessels whereas bevacizumab stimulated their development, as we described previously (M. Dufies et al., Cancer Res 77, 1212-1226 (2017); R. Grepin et al., Oncogene 31, 1683-1694 (2012)), FIG. 12H). Moreover, a 3-fold increase in the plasmatic levels of VEGF_{XXX/NF} was observed in mice treated with bevacizumab (FIG. 12 I). These results highlighted the relevance of specific VEGF_{XXX/NF} inhibition for the treatment of RCC.

### Example 9: VEGF_{222/NF} is Synonymous of Poor Prognosis for Metastatic ccRCC Patients and Predicts the Response to Sunitinib

The detrimental effects of VEGF_{222/NF} in immunodeficient mice and zebrafish models prompted us to analyze the prognostic impact of its plasmatic levels in comparison to plasmatic VEGF level on a cohort of 47 metastatic (M1) ccRCC patients treated by sunitinib. Clinical characteristics of these patients are presented in Table S3. The third quartile was used as the cut-off to determine patients' groups, respectively 4500 μg/ml and 3000 μg/ml for VEGF and VEGF_{222/NF}. The progression-free survival (PFS) was significantly reduced in the high-VEGF and the high VEGF_{222/NF} groups (FIGS. 13A,B). Hence, VEGF, as already described (Wierzbicki et al., 2019, Prognostic significance of VHL, HIF1A, HIF2A, VEGFA and p53 expression in patients with clear cell renal cell carcinoma treated with sunitinib as first line treatment, Int J Oncol 55, 371-390) and VEGF_{222/NF} should therefore be considered as equivalent predictive markers of sunitinib response. However, patients with high plasmatic levels of both VEGF_{222/NF} and VEGF had the shortest PFS (FIG. 14). This result suggests that both VEGFs' levels deserve to be tested to stratify the eligible patients to sunitinib therapy. Whereas VEGF was not associated with a significant effect on OS, high expression of VEGF_{222/NF} WAS synonymous of a shorter overall survival (OS) in M1 patients (FIGS. 13C,D).

Hence, VEGF_{222/NF} is a more robust prognostic marker as compared to VEGF.

TABLE S3
M1 ccRCC patients characteristics

| | Total | Low VEGF_{222/NF} | High VEGF_{222/NF} | p value |
|---|---|---|---|---|
| Number | 47 | 35 | 12 | |
| Age | 61.6 (30-81.3) | 63.6 (30-81.3) | 57 (43-70) | 0.0271 |
| Sex | | | | ns |
| Female | 11 (23.4%) | 9 (25.7%) | 2 (16.7%) | |
| Male | 36 (76.6%) | 26 (74.3%) | 10 (83.3%) | |
| pT | | | | 0.042 |
| ½ | 15 (31.9%) | 14 (40%) | 1 (8.3%) | |
| ¾ | 32 (68.1%) | 21 (60%) | 11 (91.7%) | |
| pN | | | | ns |
| 0 | 26 (53.2%) | 20 (57.2%) | 6 (50%) | |
| ≥1 | 6 (12.8%) | 4 (11.4%) | 2 (16.7%) | |
| x | 15 (31.9%) | 11 (31.4%) | 4 (33.3%) | |
| pM | | | | ns |
| 0 | 26 (55.3%) | 21 (60%) | 5 (41.7%) | |
| 1 | 21 (44.7%) | 14 (40%) | 7 (58.3%) | |
| Fuhrman grade | | | | ns |
| ½ | 14 (29.8%) | 13 (37.2%) | 1 (8.3%) | |
| ¾ | 33 (70.2%) | 22 (62.8%) | 11 (91.7%) | |
| PFS (months)/ | 12 | 15 | 5 | 0.0179 |
| progression % | 82% | 76% | 83% | |
| OS (months)/ | 33 | 37 | 13 | 0.0243 |
| Death % | 75% | 68% | 83% | |

1. An isolated splice variant isoform of VEGF having pro-angiogenic and pro-lymphangiogenic activity, said isoform comprising an amino acid sequence having at least 80% identity to the amino acid sequence of SEQ ID NO:1.

2. The isolated isoform of VEGF according to claim 1, said isoform comprising the amino acid sequence of SEQ ID NO:1.

3. The isolated isoform of VEGF according to claim 1, which is VEGF_{222/NF} and consists of SEQ ID NO:1.

4. The isolated isoform of VEGF according to claim 1, consisting of SEQ ID NO:2.

5. An isolated cDNA nucleotide molecule capable of encoding the isolated splice variant isoform of VEGF according to claim 1, said cDNA molecule comprising a nucleotide sequence having at least 80% identity to the nucleotide sequence of SEQ ID NO:3.

6. An isolated RNA nucleotide molecule having a sequence which is transcribed from the cDNA according to claim 5, said RNA molecule comprising a nucleotide sequence having at least 80% identity to the nucleotide sequence of SEQ ID NO:4.

7. A method of alleviating a symptom of a disease or disorder of the nervous system chosen from neurodegenerative disorders, neural stem cell disorders, neural progenitor disorders, ischemic disorders, neurological traumas, affective disorders, neuropsychiatric disorders, learning and memory disorders, Parkinson's disease and Parkinsonian disorders, Huntington's disease, Alzheimer's disease, Amyotrophic Lateral Sclerosis, spinal ischemia, ischemic stroke, spinal cord injury, cancer-related brain/spinal cord injury, schizophrenia and other psychoses, depression, bipolar depression/disorder, anxiety syndromes/disorders, phobias, stress and related syndromes, cognitive function disorders, aggression, drug and alcohol abuse, obsessive compulsive behavior syndromes, seasonal mood disorder, borderline personality disorder, cerebral palsy, life style drug, multi-infarct dementia, Lewy body dementia, age related/geriatric dementia, epilepsy and injury related to epilepsy, spinal cord injury, brain injury, trauma related brain/spinal cord injury, anti-cancer treatment related brain/spinal cord tissue injury, infection and inflammation related brain/spinal cord injury, environmental toxin related brain/spinal cord injury, multiple sclerosis, autism, attention deficit disorders, narcolepsy and sleep disorders, to stimulate the development of collateral circulation in cases of arterial and/or venous obstruction selected from myocardial infarcts, ischemic limbs, deep venous thrombosis, and/or postpartum vascular problems and to treat lymphedema post radiotherapy or Milroy disease in which the lymphatic vessel system is damaged, comprising the administration to a subject in need thereof of a pharmaceutical substance with pro-angiogenic and pro-lymphangiogenic activity comprising the isolated isoform of VEGF according to claim 1.

8. A method comprising the administration to a subject in need thereof of a pharmaceutical substance comprising an inhibitor of the pro-angiogenic and pro-lymphangiogenic activity of the isolated isoform of VEGF according to claim 1.

9. The method according to claim 8, wherein the angiogenesis-dependent disease is selected from the group of pathologies presenting exacerbated angiogenesis including tumor and metastasis, rheumatoid arthritis, atherosclerosis, neointimal hyperplasia, diabetic retinopathy and other complications of diabetes, trachoma, retrolental fibroplasia, neovascular glaucoma, age-related macular degeneration, diabetes, retinopathies, haemangiomas, immune rejection of transplanted corneal tissue, corneal angiogenesis associated with ocular injury or infection, vascular disease, obesity, psoriasis, arthritis, and gingival hypertrophy.

10. The method according to claim 8 wherein the inhibitor is chosen from an antibody, a protein, a siRNA, a shRNA, a CRISPR guide, or an antisense oligonucleotide.

11. An antibody raised against the isolated isoform of VEGF according to claim 1.

12. The antibody according to claim 11, the antibody being specific to the amino acid sequence of SEQ ID NO:1.

13. The antibody according to claim 11 or 12, the antibody being specific to the epitopes of SEQ ID NO:5 and/or SEQ ID NO:6.

14. A method of prognosing or predicting the efficacy of specific treatments comprising the use of the isolated isoform of VEGF according to claim 1, produced from expression vectors containing a cDNA molecule comprising a nucleotide sequence having at least 80%, identity to the nucleotide sequence of SEQ ID NO: 3.

15. The method according to claim 14 of prognosing non metastatic clear cell Renal Cell Carcinoma (ccRCC) in mammalian patients comprising the use the isolated isoform of VEGF for use according to claim 14.

16. The method according to claim 14, of predicting the efficacy of treatments by the compounds chosen from bevacizumab, sunitinib, ranibizumab, pegaptanib sodium, aflibercept, and brolucizumab.

17. A method of alleviating a symptom of a disease or disorder of the nervous system chosen from neurodegenerative disorders, neural stem cell disorders, neural progenitor disorders, ischemic disorders, neurological traumas, affective disorders, neuropsychiatric disorders, learning and memory disorders, Parkinson's disease and Parkinsonian disorders, Huntington's disease, Alzheimer's disease, Amyotrophic Lateral Sclerosis, spinal ischemia, ischemic stroke, spinal cord injury, cancer-related brain/spinal cord injury, schizophrenia and other psychoses, depression, bipolar depression/disorder, anxiety syndromes/disorders, phobias, stress and related syndromes, cognitive function disorders, aggression, drug and alcohol abuse, obsessive compulsive behavior syndromes, seasonal mood disorder, borderline personality disorder, cerebral palsy, life style drug, multi-infarct dementia, Lewy body dementia, age related/geriatric dementia, epilepsy and injury related to epilepsy, spinal cord injury, brain injury, trauma related brain/spinal cord injury, anti-cancer treatment related brain/spinal cord tissue injury, infection and inflammation related brain/spinal cord injury, environmental toxin related brain/spinal cord injury, multiple sclerosis, autism, attention deficit disorders, narcolepsy and sleep disorders, to stimulate the development of collateral circulation in cases of arterial and/or venous obstruction selected from myocardial infarcts, ischemic limbs, deep venous thrombosis, and/or postpartum vascular problems and to treat lymphedema post radiotherapy or Milroy disease in which the lymphatic vessel system is damaged, comprising the administration to a subject in need thereof of a pharmaceutical substance with pro-angiogenic and pro-lymphangiogenic activity comprising the isolated nucleotide molecule according to claim 5.

18. A method in the prevention and the treatment of an angiogenesis-dependent disease condition, comprising the administration to a subject in need thereof of a pharmaceutical substance comprising the isolated nucleotide molecule according to claim 5.

19. The method according to claim 18, wherein the angiogenesis-dependent disease is selected from the group of pathologies presenting exacerbated angiogenesis including tumor and metastasis, rheumatoid arthritis, atherosclerosis, neointimal hyperplasia, diabetic retinopathy and other complications of diabetes, trachoma, retrolental fibroplasia, neovascular glaucoma, age-related macular degeneration, diabetes, retinopathies, haemangiomas, immune rejection of transplanted corneal tissue, corneal angiogenesis associated with ocular injury or infection, vascular disease, obesity, psoriasis, arthritis, and gingival hypertrophy.


SEQUENCE LISTING (95 sequences)

SEQ ID NO: 1 - protein, 248 aa, Homo sapiens
  VEGF222/NF (Protein sequence)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCSLCFPFPSDVTSRGGEPGRRKEPPSGFREPDLSPGKTDTERSIQKPRCRHHTITIDRTVLNPET

SEQ ID NO: 2 - protein, 263 aa, Artificial Sequence
  SlP-HIs-3C-VEGF222/NF
MNFYKIFVFVALILAISIGQSEAGSHHHHHHSGLEVLFQGPAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCSLCFPFPSDVTSRGGEPGRRKEPPSGFREPDLSPGKTDTERSIQKPRCRHHTITIDRTVLNPET

SEQ ID NO: 3 - DNA, 747 bp, Homo sapiens
  VEGF222/NF (cDNA sequence)
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagcctttgttttccatttccctcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctga

SEQ ID NO: 4 - RNA, 747 nt, Homo sapiens
  VEGF222/NF (RNA sequence)
augaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagccuuuguuuuccauuucccucagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccuga

SEQ ID NO: 5 - protein, 14 aa, Artificial Sequence
  Epitope#1 of the VEGF222/NF
DVTSRGGEPGRRKE

SEQ ID NO: 6 - protein, 14 aa, Artificial Sequence
  Epitope#2 of the VEGF222/NF
SGFREPDLSPGKTD

SEQ ID NO: 7 - DNA, 21 bp, Artificial Sequence
  Forward primer
gcctttgttttccatttccct

SEQ ID NO: 8 - DNA, 21 bp, Artificial Sequence
  Reverse primer
tctgtcgatggtgatggtgtg

SEQ ID NO: 9 - protein, 6 aa, Artificial Sequence
  CDKPRR
CDKPRR

SEQ ID NO: 10 - protein, 6 aa, Artificial Sequence
  SLTRKD
SLTRKD

SEQ ID NO: 11 - RNA, 62 nt, Artificial Sequence
  VEGF pre-mRNA
gccucaucgccaggccuccucacuuggcccuaaccccagccuuuguuuuccauuucccucag

SEQ ID NO: 12 - protein, 63 aa, Artificial Sequence
  resulting C-terminal specific sequence of VEGF222/NF
LCFPFPSDVTSRGGEPGRRKEPPSGFREPDLSPGKTDTERSIQKPRCRHHTITIDRTVLNPET

SEQ ID NO: 13 - protein, 7 aa, Artificial Sequence
  C-terminal Fragment 1 of VEGF222/NF of Homo Sapiens (HS), Gorilla Gorilla (GG) and Pan Troglodytes (PT)
SLCFPFP

SEQ ID NO: 14 - protein, 57 aa, Artificial Sequence
  C-terminal Fragment 2 of VEGF222/NF of Homo Sapiens (HS) and Pan Troglodytes (PT)
SDVTSRGGEPGRRKEPPSGFREPDLSPGKTDTERSIQKPRCRHHTITIDRTVLNPET

SEQ ID NO: 15 - protein, 36 aa, Artificial Sequence
  C-terminal Fragment 2 of VEGF222/NF of Gorilla Gorilla (GG)
SDVTSRGGEPGRRKEPPSGFREPDLSPGKTDTERSI

SEQ ID NO: 16 - protein, 16 aa, Artificial Sequence
  C-terminal Fragment 1 of VEGF222/NF of Sus Scrofa (SS)
ASSPGPNPRPPFPVPR

SEQ ID NO: 17 - protein, 50 aa, Artificial Sequence
  C-terminal Fragment 2 of VEGF222/NF of Sus Scrofa (SS)
ADVTSRGGEPGWRKEPPSAFWAPDVSPGKTDTERPVAAATTTPPPSTEQS

SEQ ID NO: 18 - protein, 56 aa, Artificial Sequence
  C-terminal Fragment of VEGF222/NF of Canis Lupus (CL)
WGGCLPHHCVPVPGPNNPWPLIPTDVTSPGGEPGWKKEPPSGFREPDLSPGKPDSE

SEQ ID NO: 19 - protein, 25 aa, Artificial Sequence
  C-terminal Fragment 1 of VEGF222/NF of Mus Musculus (MM)
RPGEAACLHCQAPVALTPCLSLPFP

SEQ ID NO: 20 - protein, 52 aa, Artificial Sequence
  C-terminal Fragment 2 of VEGF222/NF of Mus Musculus (MM)
IDVTSQGGEPGCRKEPPSGFREPDLSPERPINHVTTTPSSSPLTEQSLIQKA

SEQ ID NO: 21 - protein, 21 aa, Artificial Sequence
  C-terminal Fragment 1 of VEGF222/NF of Rattus Norvegicus (RN)
RLLASSPGSCGPNPRLFLPFA

SEQ ID NO: 22 - protein, 52 aa, Artificial Sequence
  C-terminal Fragment 2 of VEGF222/NF of Rattus Norvegicus (RN)
TDVTSQGGEPGCRKEPPSGFRELDLSPERPINHVTTTPPSSPSTEQSLIQKA

SEQ ID NO: 23 - protein, 6 aa, Artificial Sequence
  C-terminal Fragment 1 consensus of VEGF222/NF between species
LCFPFP

SEQ ID NO: 24 - protein, 10 aa, Artificial Sequence
  C-terminal Fragment 2 consensus of VEGF222/NF between species
DVTSRGGEPG

SEQ ID NO: 25 - protein, 22 aa, Artificial Sequence
  C-terminal Fragment 3 consensus of VEGF222/NF between species
RKEPPSGFREPDLSPGKTDTER

SEQ ID NO: 26 - DNA, 21 bp, Artificial Sequence
  shScramble
cctaaggttaagtcgccctcg

SEQ ID NO: 27 - DNA, 21 bp, Artificial Sequence
  shVEGF#1
gcgcaagaaatcccggtataa

SEQ ID NO: 28 - DNA, 21 bp, Artificial Sequence
  shVEGF#2
agggcagaatcatcacgaagt

SEQ ID NO: 29 - DNA, 19 bp, Artificial Sequence
  shVEGFNF#1
gcctttgttttccatttcc

SEQ ID NO: 30 - DNA, 21 bp, Artificial Sequence
  shVEGFNF#2
catttccctcagatgtgacaa

SEQ ID NO: 31 - DNA, 21 bp, Artificial Sequence
  shNRP1
tgtggatgacattagtattaa

SEQ ID NO: 32 - DNA, 21 bp, Artificial Sequence
  shNRP2
cctcaacttcaaccctcactt

SEQ ID NO: 33 - DNA, 21 bp, Artificial Sequence
  qPCR Forward VEGF222/NF = PCR Forward VEGF222/NF
gcctttgttttccatttccct

SEQ ID NO: 34 - DNA, 21 bp, Artificial Sequence
  qPCR Reverse VEGF222/NF = PCR Reverse VEGF222/NF
tctgtcgatggtgatggtgtg

SEQ ID NO: 35 - DNA, 24 bp, Artificial Sequence
  qPCR Forward VEGF = PCR Forward VEGF
tttctgctgtcttgggtgcattgg

SEQ ID NO: 36 - DNA, 24 bp, Artificial Sequence
  qPCR Reverse VEGF = PCR Reverse VEGF
accacttcgtgatgattctgccct

SEQ ID NO: 37 - DNA, 22 bp, Artificial Sequence
  qPCR Forward VEGF121
atcttcaagccatcctgtgtgc

SEQ ID NO: 38 - DNA, 22 bp, Artificial Sequence
  qPCR Reverse VEGF121
tgcgcttgtcacatttttcttg

SEQ ID NO: 39 - DNA, 21 bp, Artificial Sequence
  qPCR Forward human 36b4
cagattggctacccaactgtt

SEQ ID NO: 40 - DNA, 20 bp, Artificial Sequence
  qPCR Reverse human 36b4
ggccaggactcgtttgtacc

SEQ ID NO: 41 - DNA, 22 bp, Artificial Sequence
  qPCR Forward mouse 36b4
agattcgggatatgctgttggc

SEQ ID NO: 42 - DNA, 21 bp, Artificial Sequence
  qPCR Reverse mouse 36b4
tcgggtcctagaccagtgttc

SEQ ID NO: 43 - DNA, 20 bp, Artificial Sequence
  qPCR Forward CD31
acgctggtgctctatgcaag

SEQ ID NO: 44 - DNA, 21 bp, Artificial Sequence
  qPCR Reverse CD31
tcagttgctgcccattcatca

SEQ ID NO: 45 - DNA, 23 bp, Artificial Sequence
  qPCR Forward VEGFR2
tttggcaaatacaacccttcaga

SEQ ID NO: 46 - DNA, 21 bp, Artificial Sequence
  qPCR Reverse VEGFR2
gcagaagatactgtcaccacc

SEQ ID NO: 47 - DNA, 21 bp, Artificial Sequence
  qPCR Forward N1SMA
gtcccagacatcagggagtaa

SEQ ID NO: 48 - DNA, 21 bp, Artificial Sequence
  qPCR Reverse N1SMA
tcggatacttcagcgtcagga

SEQ ID NO: 49 - DNA, 22 bp, Artificial Sequence
  qPCR Forward CD45
cctcacctgctcctcaaacttc

SEQ ID NO: 50 - DNA, 22 bp, Artificial Sequence
  qPCR Reverse CD45
catccactttgccctctgcttc

SEQ ID NO: 51 - DNA, 22 bp, Artificial Sequence
  qPCR Forward IL-1
gcaactgttcctgaactcaact

SEQ ID NO: 52 - DNA, 21 bp, Artificial Sequence
  qPCR Reverse IL-1
atcttttggggtccgtcaact

SEQ ID NO: 53 - DNA, 18 bp, Artificial Sequence
  qPCR Forward TNFN1
ctatgtcagcctcttctc

SEQ ID NO: 54 - DNA, 20 bp, Artificial Sequence
  qPCR Reverse TNFN1
catttgggaacttctcatcc

SEQ ID NO: 55 - DNA, 20 bp, Artificial Sequence
  qPCR Forward iNOS
tcaccttcgagggcagccga

SEQ ID NO: 56 - DNA, 20 bp, Artificial Sequence
  qPCR Reverse iNOS
tccgtggcaaagcgagccag

SEQ ID NO: 57 - DNA, 20 bp, Artificial Sequence
  qPCR Forward TGFN2
atacgcctgagtggctgtct

SEQ ID NO: 58 - DNA, 20 bp, Artificial Sequence
  qPCR Reverse TGFN2
cgctgaatcgaaagccctgt

SEQ ID NO: 59 - DNA, 20 bp, Artificial Sequence
  qPCR Forward ARG1
gattatcggagcgcctttct

SEQ ID NO: 60 - DNA, 22 bp, Artificial Sequence
  qPCR Reverse ARG1
ccacactgactcttccattctt

SEQ ID NO: 61 - DNA, 20 bp, Artificial Sequence
  qPCR Forward PD-1
accctggtcattcacttggg

SEQ ID NO: 62 - DNA, 21 bp, Artificial Sequence
  qPCR Reverse PD-1
catttgctccctctgacactg

SEQ ID NO: 63 - DNA, 22 bp, Artificial Sequence
  qPCR Forward TIM-3
tcaggtcttaccctcaactgtg

SEQ ID NO: 64 - DNA, 23 bp, Artificial Sequence
  qPCR Reverse TIM-3
ggcattcttaccaacctcaaaca

SEQ ID NO: 65 - DNA, 21 bp, Artificial Sequence
  qPCR Forward CTLA-4
ttttgtagccctgctcactct

SEQ ID NO: 66 - DNA, 21 bp, Artificial Sequence
  qPCR Reverse CTLA-4
ctgaaggttgggtcacctgta

SEQ ID NO: 67 - DNA, 19 bp, Artificial Sequence
  PCR Forward 36b4
ggcgacctggaagtccaac

SEQ ID NO: 68 - DNA, 20 bp, Artificial Sequence
  PCR Reverse 36b4
ccatcagcaccacagccttc

SEQ ID NO: 69 - DNA, 792 bp, Artificial Sequence
  SlP-HIs-3C-VEGF222/NF
atgaacttctacaagatcttcgtcttcgtcgccctgatcctggccatcagcatcggtcagagcgaagctggtagccaccaccaccaccatcacagcggtctggaagtgctgttccagggtcctgcccctatggctgagggcggtggtcagaaccaccacgaagtggtcaagttcatggacgtgtaccagcgcagctactgccaccctatcgaaactctggtggacatcttccaggagtaccctgacgagatcgagtacatcttcaagcctagctgcgtgcctctgatgcgttgcggtggttgctgcaacgacgagggtctggagtgcgtgcctactgaagagagcaacatcactatgcagatcatgcgcatcaagcctcaccagggtcagcacatcggtgaaatgagcttcctgcagcacaacaagtgcgaatgccgccctaagaaggaccgcgctcgccaggaaaacccttgcggtccttgctccgagcgtcgcaagcacctgttcgtgcaggacccccagacctgcaagtgctcctgcaagaacactgactcccgttgcaaggcccgtcagctggagctgaacgagcgcacctgcagcctgtgcttccctttccccagcgacgtcacttcccgtggcggcgagcctggtcgccgtaaggaaccccctagcggtttccgcgaacccgacctgtcccccggtaaaaccgacaccgagcgtagcatccagaagcctcgctgccgtcaccacaccatcaccatcgaccgcaccgtgctgaaccctgaaacctaa

SEQ ID NO: 70 - DNA, 20 bp, Artificial Sequence
  qPCR Forward VEGFNF total
cctttgttttccatttccct

SEQ ID NO: 71 - DNA, 21 bp, Artificial Sequence
  qPCR Reverse VEGFNF total
tctgtcgatggtgatggtgtg

SEQ ID NO: 72 - DNA, 21 bp, Artificial Sequence
  PCR Forward VEGFNF total
gcggatcaaacctcaccaagg

SEQ ID NO: 73 - DNA, 23 bp, Artificial Sequence
  PCR Reverse VEGFNF total
gcttgtcacatctgagggaaatg

SEQ ID NO: 74 - DNA, 25 bp, Artificial Sequence
  qPCR Forward hVEGF165
tgtttgtacaagatccgcagacgtg

SEQ ID NO: 75 - DNA, 26 bp, Artificial Sequence
  qPCR Reverse hVEGF165
ctcggcttgtcacatctgcaagtacg

SEQ ID NO: 76 - DNA, 23 bp, Artificial Sequence
  qPCR Forward hVEGF 168/NF
cccactgaggagtccaacatcac

SEQ ID NO: 77 - DNA, 27 bp, Artificial Sequence
  qPCR Reverse  hVEGF 168/NF
ggaaaacaaaggctgcattcacatttg

SEQ ID NO: 78 - DNA, 21 bp, Artificial Sequence
  qPCR Forward hVEGF 178/NF
gcggatcaaacctcaccaagg

SEQ ID NO: 79 - DNA, 28 bp, Artificial Sequence
  qPCR Reverse  hVEGF 178/NF
ggaaaacaaaggttttcttgtcttgctc

SEQ ID NO: 80 - DNA, 21 bp, Artificial Sequence
  qPCR Forward hVEGF 202/NF
gcggatcaaacctcaccaagg

SEQ ID NO: 81 - DNA, 30 bp, Artificial Sequence
  qPCR Reverse  hVEGF 202/NF
ggaaaacaaaggacgctccaggacttatac

SEQ ID NO: 82 - DNA, 24 bp, Artificial Sequence
  qPCR Forward hVEGF 222/NF
caagacaagaaaatccctgtgggc

SEQ ID NO: 83 - DNA, 28 bp, Artificial Sequence
  qPCR Reverse hVEGF 222/NF
ggaaatggaaaacaaaggctgcaagtac

SEQ ID NO: 84 - DNA, 22 bp, Artificial Sequence
  qPCR Forward hVEGF 240/NF
caagaaatcccgtccctgtggg

SEQ ID NO: 85 - DNA, 28 bp, Artificial Sequence
  qPCR Reverse hVEGF 240/NF
ggaaatggaaaacaaaggctgcaagtac

SEQ ID NO: 86 - DNA, 19 bp, Artificial Sequence
  qPCR Forward hVEGF 246/NF
cctggagcgttccctgtgg

SEQ ID NO: 87 - DNA, 28 bp, Artificial Sequence
  qPCR Reverse hVEGF 246/NF
ggaaatggaaaacaaaggctgcaagtac

SEQ ID NO: 88 - DNA, 21 bp, Artificial Sequence
  qPCR Forward hVEGF 263/NF
cctggagcgtgtacgttggtg

SEQ ID NO: 89 - DNA, 28 bp, Artificial Sequence
  qPCR Reverse hVEGF 263/NF
ggaaatggaaaacaaaggctgcaagtac

SEQ ID NO: 90 - DNA, 20 bp, Artificial Sequence
  qPCR Forward mVEGFR3
cgagtcggagccttctgagg

SEQ ID NO: 91 - DNA, 20 bp, Artificial Sequence
  qPCR Reverse  mVEGFR3
gcagtccagcaatagggggt

SEQ ID NO: 92 - DNA, 21 bp, Artificial Sequence
  qPCR Forward mLYVE-1
cagcacactagcctggtgtta

SEQ ID NO: 93 - DNA, 22 bp, Artificial Sequence
  qPCR Reverse mLYVE-1
cgcccatgattctgcatgtaga

SEQ ID NO: 94 - DNA, 21 bp, Artificial Sequence
  qPCR Forward mPROX1
tgcgtgttgcaccacagaata

SEQ ID NO: 95 - DNA, 22 bp, Artificial Sequence
  qPCR Reverse  mPROX1
agaagggttgacattggagtga